(19) United States
(12) Patent Application Publication
(10) Pub. No.: US 2026/0327555 A1
(43) Pub. Date: Oct. 22, 2026

(54) ENGINEERING HUMAN SKIN MICROBES TO PRODUCE MOSQUITO REPELLENT TERPENES

(71) Applicant: Massachusetts Institute of Technology, Cambridge, MA (US)

(72) Inventors: Mohamed Ibrahim HASSAN, Cambridge, MA (US); Christopher A VOIGT, Cambridge, MA (US); Geng-Min LIN, Cambridge, MA (US); Daniel YOO, Cambridge, MA (US)

(21) Appl. No.: 19/490,175

(22) PCT Filed: Apr. 5, 2024
(86) PCT No.: PCT/US2024/023221

§ 371 Date: Dec. 4, 2025

Related U.S. Application Data
(60) Provisional application No. 63/506,969, filed on Jun. 8, 2023.

(51) Int. Cl. C12N 1/20; A61K 31/015; A61K 31/045; A61K 35/74; A61P 39/00; C12N 15/77; C12R 1/15; C12R 1/45

(52) U.S. Cl. CPC C12N 1/20; A61K 31/015; A61K 31/045; A61K 35/74; A61P 39/00; C12N 15/77; C12N 2830/34; C12R 2001/15; C12R 2001/45

(57) ABSTRACT

Engineered bacterial cells for producing a mosquito-repelling terpene are provided, that include an engineered terpene biosynthetic pathway introduced into a bacterial cell that is not otherwise capable of producing the terpene, such that the engineered bacterial cell is capable of producing the mosquito-repelling terpene, and methods for using such cells to repel mosquitos.

### FEDERAL FUNDING STATEMENT

This invention was made with government support under HR0011-20-2-0030 awarded by the Defense Advanced Research Projects Agency. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Mar. 11, 2024 having the file name “23-0790-WO.xml” and is 61,465 bytes in size.

### BACKGROUND

Mosquitoes are a major source of vector-borne diseases including malaria, dengue, West Nile, zika and yellow fever, affecting millions every year. Vector-borne diseases account for 17% of all infectious diseases, resulting in >700,000 deaths annually (World Health Organization, 2022). Minimizing contact of mosquitoes is key to preventing the transmission of illnesses.

### SUMMARY

In one aspect, the disclosure provides engineered bacterial cells for producing a mosquito-repelling terpene, comprising an engineered terpene biosynthetic pathway introduced into a bacterial cell that is not otherwise capable of producing the terpene, wherein the engineered bacterial cell is capable of producing the mosquito-repelling terpene. In one embodiment the bacterial cell is a human skin microbe; in other embodiments, the bacterial cell is Corynebacterium amycolatum, Staphylococcus caprae, or Staphylococcus epidermidis.

In one embodiment, the mosquito-repelling terpene is selected from the group consisting of eucalyptol, kaur-16-ene, and kolavelool. In one embodiment, the mosquito-repelling terpene comprises eucalyptol. In one such embodiment, the bacterial cell is transformed with polynucleotides encoding the following polypeptides having enzymatic activity: acetyl-CoA C-acetyltransferase (AtoB), hydroxymethylglutaryl-CoA synthase (HMGS), hydroxymethylglutaryl-CoA reductase (HMGR), mevalonate kinase (MK), phosphomevalonate kinase (PMK), diphosphomevalonate decarboxylase (MVD), Isopentenyl-diphosphate Delta-isomerase (IDI), geranyldiphosphate synthase (GPPS), and 1,8 cineole synthase (bCinS).

In another embodiment, the mosquito-repelling terpene comprises kaur-16-ene. In one such embodiment, the bacterial cell is transformed with polynucleotides encoding the following polypeptides having enzymatic activity: wherein the bacterial cell is transformed with polynucleotides encoding the following polypeptides having enzymatic activity: acetyl-CoA C-acetyltransferase (AtoB), hydroxymethylglutaryl-CoA synthase (HMGS), hydroxymethylglutaryl-CoA reductase (HMGR), mevalonate kinase (MK), phosphomevalonate kinase (PMK), diphosphomevalonate decarboxylase (MVD), Isopentenyl-diphosphate Delta-isomerase (IDI), geranylgeranyl pyrophosphate synthase (GGPS), PtmT2 cyclase (CycA07), and Hypothetical cyclase (CycB04)

In a further embodiment, the mosquito-repelling terpene comprises kolavelool. In one such embodiment, the bacterial cell is transformed with polynucleotides encoding the following polypeptides having enzymatic activity: acetyl-CoA C-acetyltransferase (AtoB), hydroxymethylglutaryl-CoA synthase (HMGS), hydroxymethylglutaryl-CoA reductase (HMGR), mevalonate kinase (MK), phosphomevalonate kinase (PMK), diphosphomevalonate decarboxylase (MVD), Isopentenyl-diphosphate Delta-isomerase (IDI), geranylgeranyl pyrophosphate synthase (GGPS); TR51_15710 cyclase (CycA01); and terpene synthase family (CycB01).

In another aspect, the disclosure provides polynucleotides comprising the nucleotide sequence of any one of SEQ ID NO: 13, 17, and 20, recombinant expression vector comprising the polynucleotides, and recombinant host cells comprising the polynucleotide or recombinant expression vector.

In another embodiment, the disclosure provides compositions comprising a plurality of the engineered bacterial cell of any embodiment or combination of embodiments herein, and a pharmaceutically acceptable carrier. In one embodiment, the compositions are formulated for topical administration, including but not limited to a spray, a lotion, an oil, an ointment, a cream, a balm, a foam, a gel, or a salve.

In a further aspect, the disclosure provides methods to repel mosquitos, comprising contacting the skin of a subject with a plurality of the engineered bacterial cells of any preceding claim, or with the composition of any embodiment herein, thereby repelling mosquitos from skin of the subject. In another aspect, the disclosure provides methods to repel mosquitos, comprising contacting the skin of a subject with kaur-16-ene, kolavelool, or a combination thereof, thereby repelling mosquitos from skin of the subject.

### DESCRIPTION OF THE FIGURES

FIG. 1. Terpene production pipeline to produce potential mosquito repellents. (A) Outline of E. coli strain harboring biosynthetic pathway including the mevalonate pathway, a prenyl transferase and terpene cyclase genes for in vivo terpene production. This strains are cultivated in screw cap vials and analyzed by GC-MS for terpene production. B: Select list of terpenes produced in E. coli.

FIG. 2. Mosquito olfactometer set up to determine repellent terpenes (A) Overview of mosquito olfactometer. Mosquitoes are placed in the start canister prior to the initiation of the experiment. A petri dish with 1 mL of bacterial culture (after cultivation for 24 h) is placed in the stimulus box. Repellency is determined by the number of mosquitoes travelling from the start canister, through the flying tube and ultimately residing in the trap after 5 minutes. (B) E. coli strains producing eucalyptol and kaur-16-ene repel mosquitoes (box) when compared to an E. coli control. LA (lactic acid)—ammonia are used to stimulate mosquito response in the olfactometer.

FIG. 3. Engineering of a primary skin isolate to repel mosquitoes. (A) Engineered S. epidermidis LM088 (sMIH197) to contain a constitutively expressed biosynthetic pathway of eucalyptol on the genome. (B) Engineered strain successfully produces eucalyptol, as detected by GCMS. (C) sMIH197 repels mosquitoes when analyzed in mosquito olfactometer described in FIG. 2A.

FIG. 4. Engineering of a skin isolate to produce repellent kaur-16-ene. (A) Biosynthesis of kaur-16-ene utilizes two diterpene cyclases (cycA07, cycB04). Successfully engineered S. epidermidis LM088 (sMI210) to constitutively express kaur-16-ene pathway. (B) Engineered strain successfully produces kaur-16-ene, as detected by GCMS.

FIG. 5. Engineering of a primary skin isolate produce Kolavelool. Biosynthesis of kolavelool utilizes two diterpene cyclases (cycA01, cycB01). Successfully engineered S. epidermidis LM088 (sMI217) to constitutively express kolavelool pathway. (B) Engineered strain successfully produces kolavelool, as detected by GCMS

FIG. 6. Olfactometer analysis of S. epidermidis eucalyptol producer (S. epi Euc.), S. epidermidis kaur-16-ene producer (S. epi Kaur.) and S. epidermidis kolavelool producer (S. epi Kol.). Positive control: Lactic acid+Ammonia (attractant). Negative control: 2-methylbutyric acid (repellent).

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein.” “above” and “below” and words of similar import, when used in this application, shall refer to this application as a whole and not to any particular portions of this application.

All embodiments of any aspect of the disclosure can be used in combination, unless the context clearly dictates otherwise.

As used herein, “about” will mean up to plus or minus 5% of the particular value.

In one aspect, the disclosure provides engineered bacterial cells for producing a mosquito-repelling terpene, comprising an engineered terpene biosynthetic pathway introduced into a bacterial cell that is not otherwise capable of producing the terpene, wherein the engineered bacterial cell is capable of producing the mosquito-repelling terpene.

As shown in the examples that follow, the inventors have identified mosquito-repellant terpenes and heterologously expressed terpene biosynthetic pathways in human skin microbes that can successfully generate the mosquito-repellant terpenes.

As used herein, “mosquito-repellant terpene” means a terpene that deters mosquitoes from approaching or settling on a subject.

The engineered bacterial cells may be any bacteria that is not otherwise capable of producing the mosquito-repellant terpene. In one embodiment, the bacterial cells are human skin microbes. Many such human skin microbes are known and included in the present disclosure. In some embodiments, the bacterial cell may be a primary isolate from human skin. In other embodiments, the bacterial cell may be one that is available from a depository or commercially. In certain embodiments, the bacterial cells comprise Corynebacterium amycolatum, Staphylococcus caprae, or Staphylococcus epidermidis. Exemplary sources for such cells include, but are not limited to, primary isolates isolated using standard techniques (including but not limited to Staphylococcus epidermidis LM088), Corynebacterium amycolatum ATCC 49368, Staphylococcus caprae ATCC 55133, and Staphylococcus epidermidis ATCC 12228.

In certain embodiments, the mosquito-repelling terpene is selected from the group consisting of eucalyptol, kaur-16-ene, and kolavelool. The structures of these compounds are shown below.

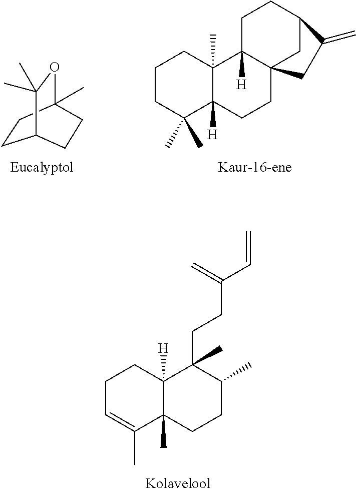

In one embodiment, the mosquito-repelling terpene comprises eucalyptol. In this embodiment, the bacterial cell is transformed with polynucleotides encoding the following polypeptides having enzymatic activity: acetyl-CoA C-acetyltransferase (AtoB), hydroxymethylglutaryl-CoA synthase (HMGS), hydroxymethylglutaryl-CoA reductase (HMGR), mevalonate kinase (MK), phosphomevalonate kinase (PMK), diphosphomevalonate decarboxylase (MVD), Isopentenyl-diphosphate Delta-isomerase (IDI), geranyldiphosphate synthase (GPPS), and 1,8 cineole synthase (bCinS). A list of the genes and encoded proteins are shown in Tables 1-2, and further details are provided in the examples.

TABLE 1
List of genes for the biosynthesis of Eucalyptol

| Gene acronym | Name | Source organism | Accession Number |
|---|---|---|---|
| AtoB | acetyl-CoA C- | Streptomyces | WP_010987600.1 |
| | acetyltransferase | | |
| HMGS | Hydroxymethylglutaryl-CoA | Streptococcus pyogenes | ABF37729.1 |
| | synthase | MGAS10750 | |
| HMGR | Hydroxymethylglutaryl-CoA | Streptococcus pyogenes | WP_002984963.1 |
| | reductase | | |
| MK | Mevalonate Kinase | Streptococcus pyogenes | WP_002984974.1 |
| PMK | Phosphomevalonate | Streptococcus pyogenes | ABF33807.1 |
| | Kinase | | |
| MVD | diphosphomevalonate | Streptococcus pyogenes | WP_011284696.1 |
| | decarboxylase | | |
| IDI | Isopentenyl-diphosphate | Streptococcus pyogenes | WP_010922169.1 |
| | Delta-isomerase | | |
| GPPS | geranyldiphosphate | Abies grandis | AAN01134.1 |
| | synthase | | |
| bCinS | 1,8 cineole synthase | Streptomyces clavuligerus | WP_003952918.1 |

In one embodiment, the bacterial cell is transformed with polynucleotides encoding acetyl-CoA C-acetyltransferase (AtoB) comprising the amino acid sequence of SEQ ID NO: 1, hydroxymethylglutaryl-CoA synthase (HMGS) comprising the amino acid sequence of SEQ ID NO:2, hydroxymethylglutaryl-CoA reductase (HMGR) comprising the amino acid sequence of SEQ ID NO:3, mevalonate kinase (MK) comprising the amino acid sequence of SEQ ID NO:4, phosphomevalonate kinase (PMK) comprising the amino acid sequence of SEQ ID NO:5, diphosphomevalonate decarboxylase (MVD) comprising the amino acid sequence of SEQ ID NO:6, Isopentenyl-diphosphate Delta-isomerase (IDI) comprising the amino acid sequence of SEQ ID NO:7, geranyldiphosphate synthase (GPPS) comprising the amino acid sequence of SEQ ID NO:8, and 1,8 cineole synthase (bCinS) comprising the amino acid sequence of SEQ ID NO:9.

TABLE 2
Amino acid sequence of encoded proteins

| Gene | |
|---|---|
| acronym | Encoded protein |
| AtoB | WP_010987600.1 |
| SEQ ID | 1 maeayiveav rtpvgrrrgg lagvhpadlg ahaltalvar |
| NO: 1 | sgidpaaved vvfgcldtvg |
| | 61 pqagdiartc wlaaglpeev pgvtvdrqcg ssqqavhfaa |
| | qavlsgtqdl vvaggvqnms |
| | 121 qipiafasrq aaeplgltqg pfagsegwra rygdlpvnqf |
| | hgaeliaakw gitrrdqeef |
| | 181 alrshrravr asdegrfdre tvaygkvtad egprrdtsle |
| | kmaalapvie ggtitaacss |
| | 241 qvsdgaaaml laseralreh gltprarvhh lsvrgedpir |
| | mlsapipata halkktalai |
| | 301 ddidlveine afapvvlawl ketgadpekv nvnggaialg |
| | hplgatgvrl mttllheler |
| | 361 tggrfglqtm cegggganvt iierl |
| HMGS | ABF37729.1 |
| SEQ ID | 1 mgktikitkr rtnmtigidk igfatsqyvl kledlalarq |
| NO: 2 | vdpakfsqgl liesfsvapi |
| | 61 tediitlaas aadqiltded rakidmvila tesstdqska |
| | saiyvhhlvg iqpfarsfev |
| | 121 kgacysataa ldyaklhvas kpdsrvlvia sdiarygvgs |
| | pgestqgsgs iallvtanpr |
| | 181 ilalnkdnva qtrdimdfwr pnysftpyvd giystkqyln |
| | clettwqayq krenlqlsdf |
| | 241 aavcfhipfp klalkglnni mdntvppehr eklieafqas |
| | ityskqigni ytgslylgll |
| | 301 sllenskvlq sgdkigffsy gsgavsefys gqlvagydkm |
| | lmtnrqalld qrtrlsvsky |
| | 361 edlfyeqvql ddngnanfdi yltgkfalta ikehqriyht |
| | ndkn |
| HMGR | WP_002984963.1 |
| SEQ ID | 1 mtktnlnwsg fskktfeerl qliekfklln aenlnqlktd |
| NO: 3 | vllpiqtanq mtenvlgrla |
| | 61 lpfsiapdfl vngstyqmpf vteepsvvaa asfaaklikr |
| | sggfkaqtln rqmigqivly |
| | 121 didqidnaka ailhktkkli alankaypsi vkrgggarti |
| | hleekgefli fyltvdtqea |
| | 181 mganmvntmm ealvpdltrl skghclmail snyateslvt |
| | tsceipvrll drdktkslql |
| | 241 aqkielasrl aqvdpyratt hnkgifngid avviatgndw |
| | raieagahay asrngsyqgl |
| | 301 sqwhfdqdkq vllgqmtlpm piaskggsig lnptvsiahd |
| | llnqpdaktl aqliasvgla |
| | 361 qnfaalkalt ssgiqaghmk lhakslalla gatqdeiapl |
| | vnalladkpi nlekahfyls |
| | 421 qlrqs |
| MK | WP_002984974.1 |
| SEQ ID | 1 mnenigygka hskiiligeh avvygypaia lpltdievvc |
| NO: 4 | hifpadkplv fdfydtlsta |
| | 61 iyaaldylqr lqepiayeiv sqvpqkrgmg ssaavsiaai |
| | ravfsycqep lsddlleilv |
| | 121 nkaeiiahtn psgldaktcl sdhaikfirn igfetieial |
| | ngyliiadtg ihghtreavn |
| | 181 kvaqfeetnl pylaklgalt qalerainqk nkvaigqlmt |
| | qahsalkaig vsiskadqlv |
| | 241 eaalragalg akmtggglgg cmialadtkd maekishrlk |
| | eegavntwiq ml |
| PMK | ABF33807.1 |
| SEQ ID | 1 makttvlsfq kqriyqmsny cvqtggklyl tgeyailipg |
| NO: 5 | qkalihfipl mmtaeispaa |
| | 61 hiqlasdmfs hkagmtpdas yaliqatvkt fadylgqsid |
| | qlepfsliit gkmerdgkkf |
| | 121 gigssgsvtl ltlkalsayy qitltpellf klaaytllkq |
| | gdngsmgdia ciayqtlvay |
| | 181 tsfdreqvsn wlqtmplkkl lvkdwgyhiq viqpalpcdf |
| | lvgwtkipai srqmiqqvta |
| | 241 sitpaflrts yqltqsamva lqeghkeelk kslagashll |
| | kelhpaiyhp klvtlvaacq |
| | 301 kqdavakssg sgggdcgial afnqdardtl iskwqeadia |
| | llyqerwgen d |
| MVD | WP_011284696.1 |
| SEQ ID | 1 mdpnvitvts yaniaiikyw gkenqakmip stssisltle |
| NO: 6 | nmftttsvsf lpdtatsdqf |
| | 61 yingvlqnde ehtkistiid qfrqpgqafv kmetqnnmpt |
| | aaglsssssg lsalvkacdq |
| | 121 lfdtqldqka laqkakfasg sssrsffgpv aawdkdsgai |
| | ykvetdlkma mimlvlnaak |
| | 181 kpissregmk lcrdtsttfd ewveqsaidy qhmltylktn |
| | nfekvgqlte analamhatt |
| | 241 ktanppfsyl tkesyqamea vkelrqegfa cyftmdagpn |
| | vkvlclekdl aqlaerlgkn |
| | 301 yriivsktkd lpdv |
| IDI | WP_010922169.1 |
| SEQ ID | 1 mtnrkddhik yalkyqspyn afddielihh slpsydlsdi |
| NO: 7 | dlsthfagqd fdfpfyinam |
| | 61 tggsqkgkav neklakvaaa tgivmvtgsy saalknpndd |
| | syrlhevadn lklatnigld |
| | 121 kpvalgqqtv qemqplflqv hvnvmqellm pegervfhtw |
| | kkhlaeyasq ipvpvilkev |
| | 181 gfgmdvnsik lahdlgiqtf disgrggtsf ayienqrggd |
| | rsylndwgqt tvqcllnaqg |
| | 241 lmdqveilas ggvrhpldmi kcfvlgarav glsrtvlelv |
| | ekyptervia ivngwkeelk |
| | 301 iimcaldckt ikelkgvdyl lygrlqqvn |
| GPPS | AAN01134.1 |
| SEQ ID | 1 maysamatmg yngmaascht lhptsplkpf hgastsleaf |
| NO: 8 | ngehmgllrg yskrklssyk |
| | 61 npasrssnat vaqlinppqk gkkavefdfn kymdskamtv |
| | nealnkaipl rypqkiyesm |
| | 121 rysllaggkr vrpvlciaac elvggteela iptacaiemi |
| | htmslmhddl pcidnddlrr |
| | 181 gkptnhkifg edtavtagna lhsyafehia vstsktvgad |
| | rilrmvselg ratgsegvmg |
| | 241 gqmvdiaseg dpsidlqtle wihihktaml lecsvvcgai |
| | iggaseivie rarryarcvg |
| | 301 llfqvvddil dvtkssdelg ktagkdlisd katypklmgl |
| | ekakefsdel lnrakgelsc |
| | 361 fdpvkaapll gladyvafrq n |
| bCinS | WP_003952918.1 |
| SEQ ID | 1 mpagheefdi pfpsrvnpfh araedrhvaw mramglitgd |
| NO: 9 | aaeatyrrws pakvgarwfy |
| | 61 laqgedldlg cdifgwffay ddhfdgptgt dprqtaafvn |
| | rtvamldpra dptgehplni |
| | 121 afhdlwqres apmsplwqrr avdhwtqylt ahiteatnrt |
| | rhtsptiady lelrhrtgfm |
| | 181 pplldlierv wraeipapvy ttpevqtllh ttngninivn |
| | dvlslekeea hgdphnlvlv |
| | 241 iqherqstrq qalatarrmi dewtdtfirt eprlpalcgr |
| | lgipladrts lytavegmra |
| | 301 airgnydwca etnryavhrp tgtgrattpw |

In all embodiments disclosed herein, the polynucleotides may be codon-optimized for expression in the bacterial cell of interest.

In all embodiments disclosed herein, the bacterial cell may be transfected with separate polynucleotides encoding each polypeptide, with a single polynucleotide encoding all of the polypeptides, or 2 or more polynucleotides each encoding 1 one or more of the polypeptides. In one embodiment, the polynucleotides are arranged in a single operon, with all polypeptides encoded by a single polynucleotide. The polynucleotides encoding the different polypeptides may be in any order in the operon. In one embodiment, the polynucleotide encoding bCinS is 5′ in the operon relative to polynucleotides encoding the other polypeptides having enzymatic activity. In a further embodiment, the operon comprises, in 5′ to 3′ order, polynucleotides encoding bCinS, atoB, HMGS, HMGR, MK, PMK, MVD, idi, and gpps.

In one embodiment, the operon comprises a heterologous promoter 5′ to the polynucleotides encoding the polypeptides. In one such embodiment, the operon comprises a heterologous promoter 5′ to the polynucleotide bCinS.

As used throughout herein, a “heterologous” promoter, insulator, or ribosome binding site is one that not naturally occurring as a regulatory sequence for the genes it is controlling expression of. Any heterologous promoter may be used as appropriate for an intended purpose. In various embodiments, the heterologous promoter comprises the nucleotide sequence of SEQ ID NO:10, 21, 22, 24, or 25. In one embodiment, the heterologous promoter comprises the nucleotide sequence of SEQ ID NO:10.

In another embodiment, the operon comprises a heterologous insulator 5′ to the polynucleotide sequence encoding bCinS. In a further embodiment, the heterologous insulator is 3′ to the heterologous promoter. Any heterologous insulator may be used as appropriate for an intended purpose. In some embodiments, the heterologous insulator comprises the nucleotide sequence of SEQ ID NO:11 or 23. In one embodiment, the heterologous insulator comprises the nucleotide sequence of SEQ ID NO:11.

In a further embodiment, the polynucleotides comprise a heterologous ribosome binding site (RBS) 5′ to all 9 of the genes encoding the polypeptides. Any heterologous RBS may be used as appropriate for an intended purpose. In one embodiment, the RBS comprises the nucleotide sequence of SEQ ID NO: 12.

TABLE 3
Sequences of regulatory elements

| | |
|---|---|
| Ppen | Cggtggaaacgaggtcatcatttccttccgaaaa |
| (promoter) | aacggttgcatttaaatcttacatatgtaatact |
| SEQ ID NO: 10 | ttcaaagactacatttgtaagatttga |
| RiboJ | Cggtggaaacgaggtcatcatttccttccgaaaa |
| (insulator) | aacggttgcatttaaatcttacatatgtaatact |
| SEQ ID NO: 11 | ttcaaagactacatttgtaagatttga |
| SocA (RBS) | TTAGGAGGATGATTATTT |
| SEQ ID NO: 12 | |
| Pveg | aattttgtcaaaataattttattgacaacgtctt |
| (Promoter) | attaacgttgatataatttaaattttatttgaca |
| (SEQ ID | aaaatgggctcgtgttgtacaataaatgt |
| NO: 21) | |
| BBa_K090504 | Atttttaaagtatgtatacaaatgatgaataaat |
| (Promoter) | tttggcgatataatgaaggatacagctcccataa |
| (SEQ ID | ttggtaaagatactacatagattcatcgtaaaat |
| NO: 22) | catgattttgccaaatttgcccttgaatattagt |
| | agcgttttctttacaatcgtaaatagtgtaaaaa |
| | agcgtgcaaacgcatgaatatcatctaaacgaga |
| | gattcacatgggaaaactgtatgtattgatcctc |
| SarJ | ttttcctcctttaaacaaaattatttgtagagca |
| (insulator) | ctgtttcggcccttttgggccaccgtcaggtcgg |
| (SEQ ID | atacacatccggcgacagtct |
| NO: 23) | |
| Pspank (BBa_ | ctcgagggtaaatgtgagcactcacaattcattt |
| K143C15) | tgcaaaagttgttgactttatctacaaggtgtgg |
| (promoter) | cataatgtgtgtaattgtgagccgataacaatt |
| SEQ ID NO: 24) | |
| P43 | attttacatttttagaaatgggcgtgaaaaaaag |
| (BBa_K143013) | cgcgcgattatgtaaaatataa |
| (promoter) | |
| SEQ ID NO: 25) | |

In a specific embodiment, the bacterial cell is transformed with the polynucleotide comprising the nucleotide sequence of SEQ ID NO:13, as shown below.

| SEQ ID NO: 13 | |
|---|---|
| LOCUS Eucalyptol_Pathw 10078 bp DNA | |
| 1 | cggtggaaac gaggtcatca tttccttccg aaaaaacggt tgcatttaaa tcttacatat |
| 61 | gtaatacttt caaagactac atttgtaaga tttgaagctg tcaccggatg tgctttccgg |
| 121 | tctgatgagt ccgtgaggac gaaacagcct ctacaaataa ttttgtttaa ttaggaggat |
| 181 | gattatttat gcctgcagct catgaagaat tcgatatacc atttccttca cgtgtaaatc |
| 241 | catttcatgc acgagcagaa gatcgtcatg ttgcttggat gagagctatg ggtttaatta |
| 301 | cgggtgatgc tgctgaagcg acttatcgtc gttggtcacc ggctaaagta ggtgcaagat |
| 361 | ggttttatct tgctcaaggt gaagatttag atttgggttg tgacattttt ggatggtttt |
| 421 | tcgcttatga tgaccatttt gatggtccga ctggcactga tccaagacaa actgcagctt |
| 481 | ttgttaatag aactgtcgca atgttagatc ctcgtgcaga tcctacaggt gaacatccat |
| 541 | taaatattgc atttcatgat ctatggcaaa gagagagtgc acctatgagt ccattatggc |
| 601 | aacgacgtgc tgttgatcac tggacacaat atttaacagc tcatattact gaagcaacta |
| 661 | atcgtacaag acatacatca cctacaattg cagattactt agaattacga catagaactg |
| 721 | gatttatgcc gccattgctt gacttaattg aaagagtatg gcgtgctgaa atacctgcgc |
| 781 | ctgtatatac gacaccagag gtgcaaacat tgctacacac aacaaatcag aatatcaaca |
| 841 | tagttaatga cgttttatct cttgagaaag aggaagcgca cggcgatcca cataacctag |
| 901 | tattagtgat tcaacatgaa agacaaagta ctagacaaca agcattagca actgcacgaa |
| 961 | gaatgattga tgaatggact gatacattca tccgtactga accaagactt ccagcattat |
| 1021 | gcggacgatt aggaatacca cttgctgatc gtacatcttt atacacagct gtcgaaggta |
| 1081 | tgcgtgctgc aattagagga aattatgatt ggtgtgctga aacaaatcgt tatgctgttc |
| 1141 | atcgtcctac aggaacggct cgtgcgacga ctccttggta attaggagga tgattattta |
| 1201 | tggctgaagc ttatattgtt gaagcagtta gaacgcctgt cggtagaaga cgaggtggat |
| 1261 | tagctggcgt acacccagct gatttaggag cacatgcttt aacagctcta gttgcacgtt |
| 1321 | caggaattga tcctgcagcg gttgaagatg ttgtatttgg ttgcttagat acagttggtc |
| 1381 | cacaggctgg tgatattgca agaacttgtt ggttagcagc aggtcttcct gaagaagtac |
| 1441 | caggagtgac agtagataga caatgtggaa gttcacaaca agctgtgcat tttgctgcac |
| 1501 | aagctgttct atcaggtact caggatttgg tggtcgctgg aggagttcaa aatatgtcac |
| 1561 | aaattccaat agcatttgct tctagacaag cagctgaacc tttaggttta acgcaaggac |
| 1621 | cattcgcagg ttctgaaggt tggcgtgctc gatatggtga tcttccagtt aatcaatttc |
| 1681 | atggcgcaga actaattgcg gcaaagtggg gaattacacg acgtgatcaa gaagaatttg |
| 1741 | cattaagatc acatagaaga gcggtacgtg catctgacga aggtagattt gaccgagaaa |
| 1801 | cggttgcgta tggtaaagtc actgcggatg agggaccacg tagagataca agtttagaaa |
| 1861 | aaatggcggc tttagcacca gtaattgaag gtggaacgat tacagcagca tgttcaagtc |
| 1921 | aagtaagtga tggcgcagct gctatgttat tggcttcaga acgtgctttg agagaacatg |
| 1981 | gattaacgcc tagagcacgt gtgcatcatc taagtgtaag aggagaagat ccaattagaa |
| 2041 | tgttatctgc acctatccct gcaacagcac acgcattaaa aaaaactgct ttagcaattg |
| 2101 | atgatataga tttagtagaa atcaatgaag cgttcgcacc tgttgtactt gcatggttaa |
| 2161 | aagagactgg agcagatcct gaaaaagtga atgtcaatgg tggcgcaatc gctttaggac |
| 2221 | atcctttagg tgctacaggt gttagattga tgacgacatt attacacgag ttagaaagaa |
| 2281 | ctggtggtcg ttttggttta caaacaatgt gtgaaggagg aggccaggcg aatgttacga |
| 2341 | tcatagaaag actttagtta ggaggatgat tatttatgac tataggtata gacaaaatag |
| 2401 | gtttcgctac atctcaatat gttttaaaac tagaagattt agcattggca cgtcaagtag |
| 2461 | atccagcaaa gttttctcaa ggtttattaa ttgaatcatt ttctgtagca cctatcacag |
| 2521 | aagatattat tacgttagca gcttctgcag cggaccaaat cctaactgat gaagatcgtg |
| 2581 | caaaaataga catggtaata ttagcaactg aaagtagtac tgaccaatca aaagcaagtg |
| 2641 | ctatttatgt tcaccatctt gtgggcattc aaccatttgc acgaagtttt gaagtaaagc |
| 2701 | aagcttgcta cagtgctaca gcagcattag attatgcaaa attacatgta gcaagtaaac |
| 2761 | cagatagtag agtattagtg atagcttcag acattgctcg ttatggagta ggatcaccag |
| 2821 | gagaaagtac acaaggaact ggtagtattg cattgttggt gacagcaaat ccacgtatct |
| 2881 | tagcattaaa taaagataat gttgctcaaa cacgtgatat aatggatttc tggcgaccaa |
| 2941 | attactcact tacaccatac gttgatggta tttatagtac taaacaatat cttaattgct |
| 3001 | tagagactac atggcaagct tatcagaaac gagaaaattt acaattaagt gattttgctg |
| 3061 | cagtctgtct tcatatccct tttcctaaat tagcattaaa gggtttaaat aacattatgg |
| 3121 | ataatactgt ccctccagag catagagaaa aactaattga agcgttccaa gcatcaatta |
| 3181 | cttattctaa acaaattggc aatatttata caggaagtct ttacttagga ttattgagtc |
| 3241 | ttttagaaaa tagtaaagtt ttacagtctg gtgacaagat cggttttttc tcatatggct |
| 3301 | caggagcagt ctctgaattt tattcaggcc aacttgttgc aggttatgat aaaatgttaa |
| 3361 | tgacaaatcg acaagcactt ttggatcaaa gaactagatt gtcagtttct aaatacgaag |
| 3421 | atttatttra tgaacaagtt caacttgatg ataatggtaa tgctaatttt gatatatatc |
| 3481 | taacaggtaa atttgctctt acagcaatca aagagcacca aagaatttac cacactaatg |
| 3541 | ataaaaatta attaggagca tgattattta tgactaaaac gaatttaaat tggtctggtt |
| 3601 | tctctaaaaa aacatttgaa gagcgtttac aattaataga aaaatttaaa ttactaaatg |
| 3661 | cagaaaacct aaatcaatta aaaacagatg ttttattacc tattcaaacg gcgaaccaga |
| 3721 | tgacagagaa tgtgttagct cgtttagcat tacctttttc aatagcgcca gactttttag |
| 3781 | ttaatggttc tacataccaa atgccatttg tcacagaaga gccttctgtg gtagctgcag |
| 3841 | caagttttgc tgcaaagtta ataaaaagaa gtggtggatt taaagcgcaa acacttaatc |
| 3901 | gtcaaatgat cggccaaatc gtgttatacg atattgatca aatcgataat gcgaaagcag |
| 3961 | ctatcttaca taaaacgaaa aaattgatag ctttggctaa caaagcatat ccatcaattg |
| 4021 | ttaaacgagg tggtggcgca agaactattc atttggaaga gaaaggtgaa ttcttaatat |
| 4081 | tttatttaac ggtagacaca caagaagcaa tgggtgctaa tatggtcaat acaatgatgg |
| 4141 | aagctttggt tccagactta actcgtttga gtaagggaca ttgtttaatg gcaatattat |
| 4201 | caaattatgc tacagaatca ctagttacaa catcttgcga aataccagtt agattattag |
| 4261 | atcgtgacaa aacaaaatct ttacaattag cacaaaaaat tgaattagca agtcgtctag |
| 4321 | ctcaagtgga tccttatcct gcaacaacac ataataaagg tatttttaat ggtatagacg |
| 4381 | ctgtcgtcat tgctactgct aacgactggc gtgcaattga agcaggtgca catgcatatg |
| 4441 | cttcacgaaa tggtagttat caaggcttaa gtcaatggca ttttgatcaa gacaaacagg |
| 4501 | ttttattagg tcaaatgaca ttacctatgc caattgcttc aaaaggaggt tcaataggct |
| 4561 | taaatcctac tgttagtatt gcacatgatt tattaaatca accagatgct aaaacactag |
| 4621 | ctcaacttat tgcaagtgta ggtttagcac aaaactttgc agcactaaaa gcgttaacta |
| 4681 | gttcaggtat ccaagcagct catatgaaat tacacgcgaa atctcttgct ttattggcag |
| 4741 | gcgctactca agatgaaatt gcaccattgg taaacgcttt gcttgctgat aaaccaatta |
| 4801 | atctagaaaa agctcacttt tatttatcac aactacgtca aagttgagtc gacctgcagg |
| 4861 | catgcaagct tggctgtttt ggttaggagg atgattattt atgaacgaga atataggtta |
| 4921 | tggaaaagca cattctaaaa ttattttaat aggtgaacat gcagtagtat atggctatcc |
| 4981 | agcaattgct cttcctctta cagatataga agtagtgtgt catatttttc cagcggataa |
| 5041 | accactagtt tttgacttct atgacacttt aagtacagca atatatgcgg cacttgatta |
| 5101 | tcttcaaaga ttgcaagaac caatagctta cgaaatagtt agtcaagtac cacagaagcg |
| 5161 | tggcatgggt agtagtgctg ctgttagtat tgcagcgatt agagctgttt tttcttattg |
| 5221 | tcaagagcct ttaagtgacg atttacttga aatcctagta aataaagcag aaataattgc |
| 5281 | acacacaaat ccttcaggat tagatgctaa aacatgctta tctgatcacg ctataaaatt |
| 5341 | tattagaaat ataggttttg agacaatcga aattgcttta aatggttatc taattattgc |
| 5401 | tgatacgggt attcatggtc atacaagaga agctgttaat aaagtcgctc agtttgaaga |
| 5461 | aacaaattta ccatatttag caaaacttgg tgcattaaca caagcattag aaagagctat |
| 5521 | caaccaaaaa aataaggtag caattggaca attaatgaca caagctcata gtgcattgaa |
| 5581 | agcaattggt gtatctatct ctaaagctga tcaattagtc gaagcagcat tacgagcagg |
| 5641 | tgcattaggt gctaaaatga caggtggtgg tctaggtggt tgtatgattg ctttagcaga |
| 5701 | tacaaaagat atggcagaaa aaatttcaca tagattaaag gaggaaggag cggtaaatac |
| 5761 | atggattcag atgttatact taggaggatg attatttatg agtaactact gtgtacaaac |
| 5821 | gggcggtaaa ttatatttaa caggagaata tgcaatactt attccaggtc aaaaagcttt |
| 5881 | aattcacttt attccattaa tgatgactgc tgaaatcagt ccagctgcac atattcaact |
| 5941 | tgcttcagat atgttttcac acaaagcggg tatgacacct gatgcaagtt atgctttaat |
| 6001 | tcaagcaaca gttaaaacat ttgcagatta cctaggacaa agtattgatc aattagaacc |
| 6061 | tttcagtctt attatcaccg gtaaaatgga acgtgatgga aaaaaattcg gtataggaag |
| 6121 | ttcaggatct gttactttat taactttaaa agcattgtca gcttattatc aaatcacatt |
| 6181 | aacaccagaa ttgttattta aattagctgc atatactttg ctaaaacagg gagataacgg |
| 6241 | ttctatggga gacatagcgt gcattgctta ccaaacatta gttgcatata catcttttga |
| 6301 | tcgtgaacaa gtgtcaaatt ggttacaaac tatgccatta aaaaaacttt tagtcaaaga |
| 6361 | ttggggatat catattcaag taattcagcc tgctttgcct tgtgatttct tagttggttg |
| 6421 | gactaagata cctgcaattt cacgtcaaat gattcagcaa gtaacggctt ctattacacc |
| 6481 | tgcgttttra cgtacgtcat atcaattaac acaatctgct atggtggctt tacaagaagg |
| 6541 | ccataaagaa gaattaaaaa aatcattggc tggagcttca cacttattaa aagaattaca |
| 6601 | ccctgctatt tatcatccta aattggttac attagttgct gcttgtcaaa aacaggatgc |
| 6661 | agtagcaaaa agtagtggaa gtggtggagg cgactgcggt attgctcttg catttaatca |
| 6721 | agatgcgcgt gatacattaa tttcaaaatg gcaagaagct gatatagctc ttttatacca |
| 6781 | agaaagatgg ggagagaatg actagttagg aggatgatta tttatggatc ctaatgtaat |
| 6841 | cacggttact tcatatgcaa atatagcaat aattaaatac tggggtaaag aaaatcaagc |
| 6901 | taaaatgatc ccttctacat cttctatttc attgacatta gaaaatatgt tcactactac |
| 6961 | ttcagtgtca tttttaccag acactgcaac ttcagatcaa ttttatataa acggtgtgtt |
| 7021 | acaaaatgat gaagaacata cgaaaatatc aacaattatt gatcaattcc gtcaaccagg |
| 7081 | acaagctttt gtaaaaatgg aaactcaaaa taacatgcct acagctgcag gattatcatc |
| 7141 | atcatcttca ggtttgtctg cattagtaaa ggcttgtgat caattgtttg atacacagct |
| 7201 | agatcaaaaa gcattagcac aaaaagcaaa atttgcgtct ggaagttcat cacgttcttt |
| 7261 | ctttggtcct gtcgcggcat gggataaaga ttctggcgca atatataaag ttgaaacgga |
| 7321 | cttgaaaatg gcgatgatta tgttagtttt aaacgcagca aaaaaaccta tttcatcacg |
| 7381 | tgaaggtatg aaattatgtc gtgatacatc aacaacattt gatgaatggg ttgaacagtc |
| 7441 | agcaattgac taccaacata tcttgacata tttaaaaaca aacaacttcg aaaaagttgg |
| 7501 | ccaattaaca gaagcgaatg cattggctat gcatgcaact acaaagacag cgaatcctcc |
| 7561 | ttttagttac ctaactaaag aatcttatca agctatggaa gctgtaaaag aattgcgtca |
| 7621 | agaaggtttt gcatgctatt ttacaatgga tgcaggacct aatgtcaaag tactttgttt |
| 7681 | agagaaagat ttagcacaat tggcagagag attgggaaaa aattatcgaa ttatagtatc |
| 7741 | aaaaacaaaa gacttgccag acgtttaatt aggaggatga ttatttatga ctaatagaaa |
| 7801 | agatgatcat attaagtatg ctcttaaata ccaatcacct tataacgctt ttgatgatat |
| 7861 | tgaattaatt catcatagtc taccttctta tgatcttagt gacattgatt tatcaactca |
| 7921 | ctttgcagga caagattttg actttccatt ttatattaac gctatgacag gaggatctca |
| 7981 | aaaaggtaaa gctgttaatg aaaaattagc aaaagttgca gctgcaacag gtattgtaat |
| 8041 | ggtaacagga tcttatagtg cagctttgaa aaatccaaat gatgattctt atagattaca |
| 8101 | cgaggttgct gacaatctta aattagctac taacattggt ttagacaagc ctgttgcttt |
| 8161 | aggtcagcag acagtgcaag aaatgcaacc tttgtttctt caagtacatg ttaacgttat |
| 8221 | gcaagaattg ttaatgccag aaggtgaaag agtttttcat acttggaaaa aacatctagc |
| 8281 | tgaatatgct agtcaaatac cagtcccagt aattcttaaa gaagtaggct ttggtatgga |
| 8341 | tgttaactct attaaattag cacatgattt gggtattcaa actttcgata taagtggtag |
| 8401 | aggtggaact agtttcgctt atatagaaaa tcaacgtggt ggcgatagat catacttgaa |
| 8461 | tgactggggt caaactacag ttcagtgtct tttaaatgca caaggcctaa tggatcaagt |
| 8521 | cgaaatatta gcatcaggtg gtgtacgtca tcctcttgat atgattaaat gttttgtttt |
| 8581 | aggcgctcgt gcagtaggat tatctagaac ggtgttggaa ttagttgaaa aatatccaac |
| 8641 | tgagagagta atcgctatag taaatggttg gaaggaagaa ttgaaaataa ttatgtgtgc |
| 8701 | attggattgc aaaacaataa aagagctaaa aggcgtggat tatttattat acggacgact |
| 8761 | tcaacaagta aattaagcca agacttaatc attaggagga tgattattta tggcttattc |
| 8821 | agcaatggct actatgggtt ataacggtat ggctgcatca tgtcatacac ttcatccaac |
| 8881 | atctccatta aaaccttttc acggtgcatc aacttcactt gaagcattta atggagaaca |
| 8941 | tatgggttta cttcgtggat atagtaaacg aaaattatct agttataaaa atcctgcatc |
| 9001 | tagatcatca aatgcaacgg ttgcacaatt attaaatcca ccacaaaaag gaaagaaagc |
| 9061 | tgtagaattt gattttaata aatatatgga tagtaaagct atgacagtta atgaagcgtt |
| 9121 | aaataaggct attcctctaa gatatccaca aaaaatttat gaaagtatgc gttattcatt |
| 9181 | attagcaggt ggtaaacgag tacgtcctgt cttatgcata gcagcttgtg aattagttgg |
| 9241 | tggtactgaa gaactagcta ttccaacagc gtgtgctatc gaaatgattc acactatgtc |
| 9301 | attgatgcat gatgatttac catgtattga taatgatgat ttaagaagag gtaaaccaac |
| 9361 | aaatcataaa atctttggtg aagacacagc tgttacagca ggcaacgcgt tacattcata |
| 9421 | tgcattcgag catatagctg ttagtactag taagactgtc ggagcggata gaatattacg |
| 9481 | tatggtatca gaattaggaa gagctactgg ttcagaaggt gttatgggag gtcaaatggt |
| 9541 | agatattgcg tcagaaggtg atccatcaat cgatttacaa acactagagt ggattcacat |
| 9601 | tcataaaaca gcgatgttct tagaatgttc agtagtttgt ggagcgatta taggtggtgc |
| 9661 | aagtgaaatc gtaattgaaa gagcgcgtcg ttacgcaaga tgtgttggct tattatttca |
| 9721 | agttgttgat gatattttag atgtaacaaa atctagtgat gaattaggta aaacagcagg |
| 9781 | taaagactta atatctgata aagcaacata cccaaaattg atgggattgg agaaagcaaa |
| 9841 | agaattctct gatgaattgc ttaatagagc aaaaggtgaa ttatcatgtt ttgatccagt |
| 9901 | taaagcagct ccacttcttg gattagcaga ttatgtagca ttcagacaaa atcggatgta |
| 9961 | aacagagaaa cgtcacactg gctcaccttc gggtgggcct ttctgcgttt atagcctaat |
| 10021 | acacctgttc gtggagaata taaaaagcca gattattaat ccggcttttt tattattt |

In another embodiment, the mosquito-repelling terpene comprises kaur-16-ene. In this embodiment, the bacterial cell is transformed with polynucleotides encoding the following polypeptides having enzymatic activity: wherein the bacterial cell is transformed with polynucleotides encoding the following polypeptides having enzymatic activity: acetyl-CoA C-acetyltransferase (AtoB), hydroxymethylglutaryl-CoA synthase (HMGS), hydroxymethylglutaryl-CoA reductase (HMGR), mevalonate kinase (MK), phosphomevalonate kinase (PMK), diphosphomevalonate decarboxylase (MVD), Isopentenyl-diphosphate Delta-isomerase (IDI), geranylgeranyl pyrophosphate synthase (GGPS), PtmT2 cyclase (CycA07), and Hypothetical cyclase (CycB04). A list of the genes and encoded proteins are shown in Tables 4-5, and further details are provided in the examples.

TABLE 4
List of genes for the biosynthesis of Kaur-16-ene in S. epidermidis

| Gene | | | |
|---|---|---|---|
| acronym | Name | Source organism | Accession Number |
| AtoB | acetyl-CoA C- | Streptomyces | WP_010987600.1 |
| | acetyltransferase | | |
| HMGS | Hydroxymethylglutaryl-CoA | Streptococcus pyogenes | ABF37729.1 |
| | synthase | MGAS10750 | |
| HMGR | Hydroxymethylglutaryl-CoA | Streptococcus pyogenes | WP_002984963.1 |
| | reductase | | |
| MK | Mevalonate Kinase | Streptococcus pyogenes | WP_002984974.1 |
| PMK | Phosphomevalonate | Streptococcus pyogenes | ABF33807.1 |
| | Kinase | | |
| MVD | diphosphomevalonate | Streptococcus pyogenes | WP_011284696.1 |
| | decarboxylase | | |
| IDI | Isopentenyl-diphosphate | Streptococcus pyogenes | WP_010922169.1 |
| | Delta-isomerase | | |
| GGPS | geranylgeranyl | Streptomyces sp. | WP_109382251.1 |
| | pyrophosphate synthase | NWU339 | |
| CycA07 | PtmT2 cyclase | Streptomyces platensis | ACO31276.1 |
| CycB04 | Hypothetical cyclase | Bradyrhizobium | WP_011084944.1 |

In one embodiment, the bacterial cell is transformed with polynucleotides encoding acetyl-CoA C-acetyltransferase (AtoB) comprising the amino acid sequence of SEQ ID NO: 1, hydroxymethylglutaryl-CoA synthase (HMGS) comprising the amino acid sequence of SEQ ID NO:2, hydroxymethylglutaryl-CoA reductase (HMGR) comprising the amino acid sequence of SEQ ID NO:3, mevalonate kinase (MK) comprising the amino acid sequence of SEQ ID NO:4, phosphomevalonate kinase (PMK) comprising the amino acid sequence of SEQ ID NO:5, diphosphomevalonate decarboxylase (MVD) comprising the amino acid sequence of SEQ ID NO:6, Isopentenyl-diphosphate Delta-isomerase (IDI) comprising the amino acid sequence of SEQ ID NO:7; geranylgeranyl pyrophosphate synthase (GGPS) comprising the amino acid sequence of SEQ ID NO:14; PtmT2 cyclase (CycA07) comprising the amino acid sequence of SEQ ID NO:15; and Hypothetical cyclase (CycB04) comprising the amino acid sequence of SEQ ID NO:16.

TABLE 5
Amino acid sequence of encoded proteins

| Gene | | |
|---|---|---|
| acronym | Encoded protein | |
| GGPS | WP_109382251.1 | |
| (SEQ ID | 1 | magtawdtdi pplisvrddv davledfvdm |
| NO: 14) | | qeraalgpem aplfttvrrl lfsngkrlrp |
| | 61 | llcvagwqaa gapgdshpvl rvaaalelfq |
| | | tfalihddvm ddsdtrrgrp sahralaaey |
| | 121 | lsgggrlska eahgrgaail lgdlllvwsd |
| | | emlagaglga raqarvlplv dsmraelvyg |
| | 181 | qyldllstdr msgnveaalr vtryktgkyt |
| | | verplhlgvc laggdprllq tctdfaiplg |
| | 241 | eafqlrddll nvfgdpvqtg kpvgddirag |
| | | katvllalav qdasgadlkl lstlvgspel |
| | 301 | gsedidrvrt vmettgarqr vekmitarrd |
| | | talatleead lprdvaktlr qivwmaterq |
| | 361 | s |
| CycA07 | ACO31276.1 | |
| (SEQ ID | 1 | mlevpaqptp apreaeaaal laatvadpwg |
| NO: 15) | | lvapsvydta rlvslapwld ghrerlgylv |
| | 61 | keqnqdgswg apdgyglvpt lsaveallte |
| | | lartdsgaph pphddlaaac agglgalqdg |
| | 121 | llagpvpdri gvefvapsll adintrlaal |
| | | teqapgklga wsgttltspa pdldgallag |
| | 181 | vremteqapl peklwhtlea itrdgtrgar |
| | | phegapphng svgcspaata awlgaspdpa |
| | 241 | apgvaylrdv qarfggpvps itpivyfeqa |
| | | wvlnslaasg lryeapaall dsleagltde |
| | 301 | giaaapglps dsddtaavlf alaqhgrthr |
| | | pdslmhfrrd gyfscfgver tpststnahi |
| | 361 | lealghhvrv rpddagryga eirmisdwll |
| | | dnqlpdgswm dkwhaspyya taccalalae |
| | 421 | fggpsaraav draaawalet qradgswgrw |
| | | qgtteetaym vqllmrtrtp gspgtvarsa |
| | 481 | argcdallah ddpasypglw hdkdiyapvt |
| | | viraarlaal alggaesaas gga |
| CycB04 | WP_011084944.1 | |
| (SEQ ID | 1 | miqteravqq vlewgrsltg fadehaveav |
| NO: 16) | | rggqyilqri hpslrgtsar tgrdpqdetl |
| | 61 | ivtfyrelal lfwlddcndl glispeqlaa |
| | | veqalgqgvp calpgfegca vlraslatla |
| | 121 | ydrrdyaqll ddtrcysaal raghaqavaa |
| | | erwsyaeylh ngidsiayan vfcclsllwg |
| | 181 | ldmatlrarp afrqvlrlis aigrlqndlh |
| | | gcdkdrsage adnavilllq rypampvvef |
| | 241 | lndelaghtr mlhrvmaeer fpapwgplie |
| | | amaairvqyy rtstsryrsd avrggqrapa |

In one embodiment, the polynucleotides may be codon-optimized for expression in the bacterial cell of interest. In other embodiments, the bacterial cell may be transfected with separate polynucleotides encoding each polypeptide, with a single polynucleotide encoding all of the polypeptides, or 2 or more polynucleotides each encoding 1 one or more of the polypeptides. In one embodiment, the polynucleotides are arranged in a single operon, with all polypeptides encoded by a single polynucleotide.

In one such embodiment, the polynucleotides encoding CycA07 and CycB04 are 5′ in the operon relative to polynucleotides encoding the other polypeptides having enzymatic activity. In another embodiment, the operon comprises, in 5′ to 3′ order, polynucleotides encoding CycA07, CycB04, atoB, HMGS, HMGR, MK, PMK, MVD, PMD, idi, and ggps.

In one embodiment, the operon comprises a heterologous promoter 5′ to the polynucleotides encoding the polypeptides. In one such embodiment, the operon comprises a heterologous promoter 5′ to the polynucleotide CycA07.

Any heterologous promoter may be used as appropriate for an intended purpose. In various embodiments, the heterologous promoter comprises the nucleotide sequence of SEQ ID NO: 10, 21, 22, 24, or 25. In one embodiment, the heterologous promoter comprises the nucleotide sequence of SEQ ID NO: 10.

In another embodiment, the operon comprises a heterologous insulator 5′ to the polynucleotide sequence encoding CycA07. In a further embodiment, the heterologous insulator is 3′ to the heterologous promoter. Any heterologous insulator may be used as appropriate for an intended purpose. In some embodiments, the heterologous insulator comprises the nucleotide sequence of SEQ ID NO:11 or 23. In one embodiment, the heterologous insulator comprises the nucleotide sequence of SEQ ID NO:11.

In a further embodiment, the polynucleotides comprise a heterologous ribosome binding site (RBS) 5′ to all 10 of the genes encoding the polypeptides. Any heterologous RBS may be used as appropriate for an intended purpose. In one embodiment, the RBS comprises the nucleotide sequence of SEQ ID NO:12.

In another embodiment, the bacterial cell is transformed with the polynucleotide comprising the nucleotide sequence of SEQ ID NO:17, as shown below.

| SEQ ID NO: 17 | |
|---|---|
| LOCUS Kaur-16-ene_Path 11551 bp DNA | |
| 1 | cggtggaaac gaggtcatca tttccttccg aaaaaacggt tgcatttaaa tcttacatat |
| 61 | gtaatactut caaagactac atttgtaaga tttgaagctg tcaccggatg tgctttccgg |
| 121 | tctgatgagt ccgtgaggac gaaacagcct ctacaaataa ttttgtttaa ttaggaggat |
| 181 | gattatttat gctagaggta ccagctcaac ctacacctgc tccaagagag gctgaggcag |
| 241 | ccgctttgct tgccgcaaca gtagcagacc cgtggggttt ggtggccccg tctgtatatg |
| 301 | atacggcacg attagtcact ttagctcctt ggttagacgg gcatagagag agattaggat |
| 361 | atttagtaaa ggagcaaaac caagacggta gttggggagc accagatgga tacggattag |
| 421 | taccgacact atcagcagta gaggctttat taacagagtt ggctagaaca gactcaggcg |
| 481 | caccacaccc acctcatgac gacctagcag ccgcatgtgc tggtggctta ggggctctac |
| 541 | aagacggttt gttagcagct cctgtgccag acacaattgg agtagaattc gtggcaccat |
| 601 | ctcttttagc agacattaat actagactag cagccttaac tgagcaggca ccgggtaagt |
| 661 | taggtgcttg gtcaggcact acattaactt ctcctgcacc agacttagac ggtgcgttac |
| 721 | tagcaggtgt acgagaaatg acagaacaag cacctttacc tgaaaagctt tggcacactt |
| 781 | tagaggcaat tacacgtgac ggcacaagag gcgctcgtcc acacgaggga gctccacctc |
| 841 | ataacggtag tgtgggatcc tcacctgctg caactgcggc ttggctagga gcctcaccag |
| 901 | acccagctgc acctggggtt gcttatttaa gagacgtcca agcgagattc ggtggacctg |
| 961 | tcccatctat aacacctata gtttatttcg aacaagcttg ggttttaaac tctcttgcag |
| 1021 | ctagtggttt acgttacgag gcaccagctg ctcttctaga ttcacttgag gcgggactta |
| 1081 | cagacgaggg tatagcagct gctcctggtc ttccatcaga ttcagatgac acagcggctg |
| 1141 | tcttatttgc attagcacaa catggacgaa ctcaccgtcc ggatagtcta atgcatttta |
| 1201 | gaagagacgg ttacttcact tgcttcggag tggaacgtac tccgagtaca tctactaatg |
| 1261 | cacacattct agaggcttta ggccatcacg tgactgttag accagatgac gcaggacgat |
| 1321 | acggagctga gattcgaatg atcagtgatt ggttactaga caaccagctt cctgacggtt |
| 1381 | catggatgga caagtggcat gctagtcctt actatgctac agcttgttgc gcattagcct |
| 1441 | tagcagagct cggtggtcca tcagcacgag cagctgtaga cagagcagct gcatgggcgt |
| 1501 | tagagacgca gcgagcagac ggttcatggg gacgttggca aggtacgact gaggagactg |
| 1561 | cttatatggt acaactactt atgcgtacac gaactccagg atctcctgga acagtagcac |
| 1621 | gttcagccgc tagaggttgc gacgcgttac ttgcgcacga cgaccctgca tcatatcctg |
| 1681 | gtttatggca cgacaaggat atatacgcac cagtcactgt tataagagca gcgcgattgg |
| 1741 | cagctttagc gcttggtgga gctgaaagtg cggcttctgg tggagcttga ttaggaggat |
| 1801 | gattatttat gatacaaaca gaaagagcag ttcaacaagt acttgagtgg ggtagatctt |
| 1861 | taacgggctt tgctgacgag catgcggttg aagccgtgcg tggtggacaa tatatcttgc |
| 1921 | agagaataca cccaagtttg agaggaactt ctgcgcgtac tggacgtgac cctcaggacg |
| 1981 | agacattaat agtaacattt taccgtgagt tggcattact tttctggtta gatgattgca |
| 2041 | atgacttagg ccttatatca cctgagcagt tggctgctgt ggagcaagct cttggtcagg |
| 2101 | gagtcccttg tgcactacca ggtttcgagg gatgcgctgt tcttcgtgcc tcattagcaa |
| 2161 | cattggcata cgaccgtcgt gattacgcac agttattaga tgacactcgt tgctattcag |
| 2221 | cagcattacg tgctggacat gcgcaggcag ttgcagctga aagatggagt tacgcagagt |
| 2281 | atttacataa cggtattgat agtatcgctt atgcgaatgt tttctgttgc ctttcattgt |
| 2341 | tatggggatt agacatggca acattaagag cacgaccagc cttccgtcag gtgttaagac |
| 2401 | ttatttcagc gattggaaga ttacagaacg atttgcatgg atgtgataag gatcgtagtg |
| 2461 | caggagaagc tgacaacgcg gtaatattat tactacaacg ttatcctgct atgccagtgg |
| 2521 | ttgagttcct gaatgacgag cttgctggtc acacacgtat gttacataga gtaatggcag |
| 2581 | aagaaagatt ccctgctcct tggggtccat tgattgaggc aatggctgca atacgtgtcc |
| 2641 | agtactacag aactagtaca tcaagatacc gttcagacgc tgttcgaggc ggtcaacgtg |
| 2701 | cacctgctta attaggagga tgattattta tggctgaagc ttatattgtt gaagcagtta |
| 2761 | gaacgcctgt cggtagaaga cgaggtggat tagctggcgt acacccagct gatttaggag |
| 2821 | cacatgctut aacagctcta gttgcacgtt caggaattga tcctgcagcg gttgaagatg |
| 2881 | ttgtatttgg ttgcttagat acagttggtc cacaggctgg tgatattgca agaacttgtt |
| 2941 | ggttagcagc aggtcttcct gaagaagtac caggagtgac agtagataga caatgtggaa |
| 3001 | gttcacaaca agctgtgcat tttgctgcac aagctgttct atcaggtact caggatttgg |
| 3061 | tggtcgctgg aggagttcaa aatatgtcac aaattccaat agcatttgct tctagacaag |
| 3121 | cagctgaacc tttaggttta acgcaaggac cattcgcagg ttctgaaggt tggcgtgctc |
| 3181 | gatatggtga tcttccagtt aatcaatttc atggcgcaga actaattgcg gcaaagtggg |
| 3241 | gaattacacg acgtgatcaa gaagaatttg cattaagatc acatagaaga gcggtacgtg |
| 3301 | catctgacga aggtagattt gaccgagaaa cggttgcgta tggtaaagtc actgcggatg |
| 3361 | agggaccacg tagagataca agtttagaaa aaatggcggc tttagcacca gtaattgaag |
| 3421 | gtggaacgat tacagcagca tgttcaagtc aagtaagtga tggcgcagct gctatgttat |
| 3481 | tggcttcaga acgtgctttg agagaacatg gattaacgcc tagagcacgt gtgcatcatc |
| 3541 | taagtgtaag aggagaagat ccaattagaa tgttatctgc acctatccct gcaacagcac |
| 3601 | acgcattaaa aaaaactgct ttagcaattg atgatataga tttagtagaa atcaatgaag |
| 3661 | cgttcgcacc tgttgtactt gcatggttaa aagagactgg agcagatcct gaaaaagtga |
| 3721 | atgtcaatgg tggcgcaatc gctttaggac atcctttagg tgctacaggt gttagattga |
| 3781 | tgacgacatt attacacgag ttagaaagaa ctggtggtcg ttttggttta caaacaatgt |
| 3841 | gtgaaggagg aggccaggcg aatgttacga tcatagaaag actttagtta ggaggatgat |
| 3901 | tatttatgac tataggtata gacaaaatag gtttcgctac atctcaatat gttttaaaac |
| 3961 | tagaagattt agcattggca cgtcaagtag atccagcaaa gttttctcaa ggtttattaa |
| 4021 | ttgaatcact ttctgtagca cctatcacag aagatattat tacgttagca gcttctgcag |
| 4081 | cggaccaaat cctaactgat gaagatcgtg caaaaataga catggtaata ttagcaactg |
| 4141 | aaagtagtac tgaccaatca aaagcaagtg ctatttatgt tcaccatctt gtgggcattc |
| 4201 | aaccatttgc acgaagtttt gaagtaaagc aagcttgcta cagtgctaca gcagcattag |
| 4261 | attatgcaaa attacatgta gcaagtaaac cagatagtag agtattagtg atagcttcag |
| 4321 | acattgctcg ttatggagta ggatcaccag gagaaagtac acaaggaagt ggtagtattg |
| 4381 | cattgttggt gacagcaaat ccacgtatct tagcattaaa taaagataat gttgctcaaa |
| 4441 | cacgtgatat aatggatttc tggcgaccaa attactcatt tacaccatac gttgatggta |
| 4501 | tttatagtac taaacaatat cttaattgct tagagactac atggcaagct tatcagaaac |
| 4561 | gagaaaattt acaattaact gattttgctg cagtctgttt tcatatccct tttcctaaat |
| 4621 | tagcattaaa gggtttaaat aacattatgg ataatactgt ccctccagag catagagaaa |
| 4681 | aactaattga agcgttccaa gcatcaatta cttattctaa acaaattggc aatatttata |
| 4741 | caggaagtct ttacttagga ttattgagtc ttttagaaaa tagtaaagtt ttacagtctg |
| 4801 | gtgacaagat cggttttttc tcatatggct caggagcagt ctctgaattt tattcaggcc |
| 4861 | aacttgttgc aggttatgat aaaatgttaa tgacaaatcg acaagcactt ttggatcaaa |
| 4921 | gaactagatt gtcagtttct aaatacgaag atttatttta tgaacaagtt caacttgatg |
| 4981 | ataatggtaa tgctaatttt gatatatata taacaggtaa atttgctctt acagcaatca |
| 5041 | aagagcacca aagaatttac cacactaatg ataaaaatta attaggagga tgattattta |
| 5101 | tgactaaaac gaatttaaat tggtctggtt tctctaaaaa aacatttgaa gagcgtttac |
| 5161 | aattaataga aaaatttaaa ttactaaatg cagaaaactt aaatcaatta aaaacagatg |
| 5221 | ttttattacc tattcaaacg gcgaaccaga tgacagagaa tgtgttaggt cgtttagcat |
| 5281 | tacctttttc aatagcgcca gactttttag ttaatggttc tacataccaa atgccatttg |
| 5341 | tcacagaaga gccttatgtg gtagctgcag caagttttgc tgcaaagtta ataaaaagaa |
| 5401 | gtggtggatt taaagcgcaa acacttaatc gtcaaatgat cggccaaatc gtgttatacg |
| 5461 | atattgatca aatcgataat gcgaaagcag ctatcttaca taaaacgaaa aaattgatag |
| 5521 | ctttggctaa caaagcatat ccatcaattg ttaaacgagg tggtggcgca agaactattc |
| 5581 | atttggaaga gaaaggtgaa ttcttaatat tttatttaac ggtagacaca caagaagcaa |
| 5641 | tgggtgctaa tatggtcaat acaatgatgg aagctttggt tccagactta actcgtttga |
| 5701 | gtaagggaca ttgtttaatg gcaatattat caaattatgc tacagaatca ctagttacaa |
| 5761 | catcttgcga aataccagtt agattattag atcgtgacaa aacaaaatct ttacaattag |
| 5821 | cacaaaaaat tgaattagca agtcgtctag ctcaagtgga tccttatcgt gcaacaacac |
| 5881 | ataataaagg tatttttaat ggtatagacg ctgtcgtcat tgctactggt aacgactggc |
| 5941 | gtgcaattga agcaggtgca catgcatatg cttcacgaaa tggtagttat caaggcttaa |
| 6001 | gtcaatggca ttttgatcaa gacaaacagg ttttattagg tcaaatgaca ttacctatgc |
| 6061 | caattgcttc aaaaggagct tcaataggct taaatcctac tgttagtatt gcacatgatt |
| 6121 | tattaaatca accagatgct aaaacactag ctcaacttat tgcaagtgta ggtttagcac |
| 6181 | aaaactttgc agcactaaaa gcgttaacta gttcaggtat ccaagcaggt catatgaaat |
| 6241 | tacacgcgaa atctcttgct ttattggcag gcgctactca agatgaaatt gcaccattgg |
| 6301 | taaacgctct gcttgctgat aaaccaatta atctagaaaa agctcacttt tatttatcac |
| 6361 | aactacgtca aagttgagtc gacctgcagg catgcaagct tggctgtttt ggttaggagg |
| 6421 | atgattattt atgaasgaga atataggtta tggaaaagca cattctaaaa ttattttaat |
| 6481 | aggtgaacat gcagtagtat atggctatcc agcaattgct cttcctctta cagatataga |
| 6541 | agtagtgtgt catatttttc cagcggataa accactagtt tttgacttct atgacacttt |
| 6601 | aagtacagca atatatgcgg cacttgatta tcttcaaaga ttgcaagaac caatagctta |
| 6661 | cgaaatagct agtcaagtac cacagaagcg tggcatgggt agtagtgctg ctgttagtat |
| 6721 | tgcagcgatt agagctgttt tttcttattg tcaagagcct ttaagtgacg atttacttga |
| 6781 | aatcctagta aataaagcag aaataattgc acacacaaat ccttcaggat tagatgctaa |
| 6841 | aacatgctta tctgatcacg ctataaaatt tattagaaat ataggttttg agacaatcga |
| 6901 | aattgcttta aatggttatc taattattgc tgatacgggt attcatggtc atacaagaga |
| 6961 | agctgttaat aaagtcgctc agtttgaaga aacaaattta ccatatttag caaaacttgg |
| 7021 | tgcattaaca caagcattag aaagagctat caaccaaaaa aataaggtag caattggaca |
| 7081 | attaatgaca caagctcata gtgcattgaa agcaattggt gtatctatct ctaaagctga |
| 7141 | tcaattagtc gaagcagcat tacgagcagg tgcattaggt gctaaaatga caggtggtgg |
| 7201 | tctaggtggt tgtatgattg ctttagcaga tacaaaagat atggcagaaa aaatttcaca |
| 7261 | tagattaaag gaggaaggag cggtaaatac atggattcag atgttatagt taggaggatg |
| 7321 | attatttatg agtaactact gtgtacaaac gggcggtaaa ttatatttaa caggagaata |
| 7381 | tgcaatacct attccaggtc aaaaagcttt aattcacttt attccattaa tgatgactgc |
| 7441 | tgaaatcagt ccagctgcac atattcaact tgcttcagat atgttttcac acaaagcggg |
| 7501 | tatgacacct gatgcaagtt atgctttaat tcaagcaaca gttaaaacat ttgcagatta |
| 7561 | cctaggacaa agtattgatc aattagaacc tttcagtctt attatcacgg gtaaaatgga |
| 7621 | acgtgatgga aaaaaattcg gtataggaag ttcaggatct gttactttat taactttaaa |
| 7681 | agcattgtca gcttattatc aaatcacatt aacaccagaa ttgttattta aattagctgc |
| 7741 | atatacttug ctaaaacacg gagataacgg ttctatggga gacatagcgt gcattgctta |
| 7801 | ccaaacatta gttgcatata catcttttga tcgtgaacaa gtgtcaaatt ggttacaaac |
| 7861 | tatgccatta aaaaaacttt tagtcaaaga ttggggatat catattcaag taattcagcc |
| 7921 | tgctttgcct tgtgatttct tagttggttg gactaagata cctgcaattt cacgtcaaat |
| 7981 | gattcagcaa gtaacggctt ctattacacc tgcgttttta cgtacgtcat atcaattaac |
| 8041 | acaatctgct atggtggctt tacaagaagg ccataaagaa gaattaaaaa aatcattggc |
| 8101 | tggagcttca cacttattaa aagaattaca ccctgctatt tatcatccta aattggttac |
| 8161 | attagttgct gcttgtcaaa aacaggatgc agtagcaaaa agtagtggaa gtggtggagg |
| 8221 | cgactgcggt attgctcttg catttaatca agatgcgcgt gatacattaa tttcaaaatg |
| 8281 | gcaagaagct gatatagctc ttttatacca agaaagatgg ggagagaatg actagttagg |
| 8341 | aggatgatta tttatggatc ctaatgtaat cacggttact tcatatgcaa atatagcaat |
| 8401 | aattaaatac tggggtaaag aaaatcaagc taaaatgatc ccttctacat cttctatttc |
| 8461 | attgacatta gaaaatatct tcactactac ttcagtgtca tttttaccag acactgcaac |
| 8521 | ttcagatcaa ttttatataa acggtgtgtt acaaaatgat gaagaacata cgaaaatatc |
| 8581 | aacaattact gatcaattcc gtcaaccagg acaagctttt gtaaaaatgg aaactcaaaa |
| 8641 | taacatgcct acagctgcag gattatcatc atcatcttca ggtttgtctg cattagtaaa |
| 8701 | ggcttgtgat caattgtttg atacacagct agatcaaaaa gcattagcac aaaaagcaaa |
| 8761 | atttgcgtct ggaagttcat cacgttcttt ctttggtcct gtcgcggcat gggataaaga |
| 8821 | ttctggcgca atatataaag ttgaaacgga cttgaaaatg gcgatgatta tgttagtttt |
| 8881 | aaacgcagca aaaaaaccta tttcatcacg tgaaggtatg aaattatgtc gtgatacatc |
| 8941 | aacaacattt gatgaatggg ttgaacagtc agcaattgac taccaacata tgttgacata |
| 9001 | tttaaaaaca aacaacttcg aaaaagttgg ccaattaaca gaagcgaatg cattggctat |
| 9061 | gcatgcaact acaaagacag cgaatcctcc ttttagttac ctaactaaag aatcttatca |
| 9121 | agctatggaa gctgtaaaag aattgcgtca agaaggtttt gcatgctatt ttacaatgga |
| 9181 | tgcaggacct aatgtcaaag tactttgttt agagaaagat ttagcacaat tggcagagag |
| 9241 | attgggaaaa aattatcgaa ttatagtatc aaaaacaaaa gacttgccag acgtttaatt |
| 9301 | aggaggatga ttatttatga ctaatagaaa agatgatcat attaagtatg ctcttaaata |
| 9361 | ccaatcacct tataacgctt ttgatgatat tgaattaatt catcatagtc taccttctta |
| 9421 | tgatcttagt gacattgatt tatcaactca ctttgcagga caagattttg actttccatt |
| 9481 | ttatattaac gctatgacag gaggatctca aaaaggtaaa gctgttaatg aaaaattagc |
| 9541 | aaaagttgca gctgcaacag gtattgtaat ggtaacagga tcttatagtg cagctttgaa |
| 9601 | aaatccaaat gatgattctt atagattaca cgaggttgct gacaatctta aattagctac |
| 9661 | taacattggt ttagacaagc ctgttgcttt aggtcagcag acagtgcaag aaatgcaacc |
| 9721 | tttgtttctt caagtacatg ttaacgttat gcaagaattg ttaatgccag aaggtgaaag |
| 9781 | agtttttcat acttggaaaa aacatctagc tgaatatgct agtcaaatac cagtcccagt |
| 9841 | aattcttaaa gaagtaggct ttggtatgga tgttaactct attaaattag cacatgattt |
| 9901 | gggtattcaa actttagata taagtggtag aggtggaact agtttcgctt atatagaaaa |
| 9961 | tcaacgtggt ggcgatagat catacttgaa tgactggggt caaactacag ttcagtgtct |
| 10021 | tttaaatgca caaggcctaa tggatcaagt cgaaatatta gcatcaggtg gtgtacgtca |
| 10081 | tcctcttgat atgattaaat gttttgtttt aggcgctcgt gcagtaggat tatctagaac |
| 10141 | ggtgttggaa ttagttgaaa aatatccaac tgagagagta atcgctatag taaatggttg |
| 10201 | gaaggaagaa ttgaaaataa ttatgtgtgc attggattgc aaaacaataa aagagctaaa |
| 10261 | aggcgtggat tatttattat acggacgact tcaacaagta aattaagcga agacttaatc |
| 10321 | attaggagga tgattattta tggcaggaac ggcatgggat actgatatac caacgcttat |
| 10381 | ttcagtcaga gatgatgtgg atgcagtact tgaggacttt gttgatgttc aagagagagc |
| 10441 | ggcacacgga ccagagatcg ctccattgtt cacaacggct agaagacttt tgtttagtaa |
| 10501 | cggtaaaaga ctaagaccat tattatgcgt ggcggggtgg caagcagctg gagctccagg |
| 10561 | tgacccgcat gctgtcttgc gtgttgcagc agcattagaa ctattccaga cattcgcgct |
| 10621 | aatacatgac gacgtaatgg atgacagtga tactagaaga ggtagaccta gtgcacatag |
| 10681 | agctttggca gcagagtaca tgagtggcgg tggacgatta agtaaggctg aagcgcatgg |
| 10741 | cagaggagct gccattttct taggtgacct tttattagtc tggtctgacg aaatgcttgc |
| 10801 | tggtgcgggc cttgaggcca gagcacgagc tagagtactt ccattagttg actctatgag |
| 10861 | agctgagtta gtatacggtc agtacttaga tttattatct actgatagaa tgtcaggtga |
| 10921 | cgttgaagcg gcgctaagag tagcaagata taagacaggc aagtatacag ttgaaagacc |
| 10981 | acttcatttg ggtgttagtt tagcgggcgg tgacccacga ttattacaga cgtgtacgga |
| 11041 | ttttgcattg ccattaggtg aagcattcca acttcgtgat gatcttttaa acgtctttgg |
| 11101 | ggatccagct cgtactggta agccggttgg tgacgatata cgtgaaggta aagctacagt |
| 11161 | attgttagct cttgctatac aggacgcgtc agaagctgac ttaaaattat taggtacttt |
| 11221 | agttggtagt cctgagttag gtagtgaaga tatagaccga ttcagaacag taatggagac |
| 11281 | tacaggtgca cgtcaacgtg tagaggacat gataatcgca cgtagagaca ctgcacttgc |
| 11341 | aactttggaa gaagcggatt taccacagga tgtagcagag acattaagac agatcgtttg |
| 11401 | gatggcgacg gagagacaat cataacggat gtaaacagag aaacgtcaca ctggctcacc |
| 11461 | ttcgggtggg cctttctgcg tttatagcct aatacacctg ttcgtggaga atataaaaag |
| 11521 | ccagattact aatccggctt ttttattatt t |

In a further embodiment, the mosquito-repelling terpene comprises kolavelool. In one such embodiment, the bacterial cell is transformed with polynucleotides encoding the following polypeptides having enzymatic activity: acetyl-CoA C-acetyltransferase (AtoB), hydroxymethylglutaryl-CoA synthase (HMGS), hydroxymethylglutaryl-CoA reductase (HMGR), mevalonate kinase (MK), phosphomevalonate kinase (PMK), diphosphomevalonate decarboxylase (MVD), Isopentenyl-diphosphate Delta-isomerase (IDI), geranylgeranyl pyrophosphate synthase (GGPS); TR51_15710 cyclase (CycA01); and terpene synthase family (CycB01). A list of the genes and encoded proteins are shown in Tables 6-7, and further details are provided in the examples.

TABLE 6
List of genes for the biosynthesis of Kolavelool.

| Gene | | | |
|---|---|---|---|
| acronym | Name | Source organism | Accession Number |
| AtoB | acetyl-CoA C- | Streptomyces | WP_010987600.1 |
| | acetyltransferase | | |
| HMGS | Hydroxymethylglutaryl-CoA | Streptococcus pyogenes | ABF37729.1 |
| | synthase | MGAS10750 | |
| HMGR | Hydroxymethylglutaryl-CoA | Streptococcus pyogenes | WP_002984963.1 |
| | reductase | | |
| MK | Mevalonate Kinase | Streptococcus pyogenes | WP_002984974.1 |
| PMK | Phosphomevalonate | Streptococcus pyogenes | ABF33807.1 |
| | Kinase | | |
| MVD | diphosphomevalonate | Streptococcus pyogenes | WP_011284696.1 |
| | decarboxylase | | |
| IDI | Isopentenyl-diphosphate | Streptococcus pyogenes | WP_010922169.1 |
| | Delta-isomerase | | |
| GGPS | geranylgeranyl | Streptomyces sp. | WP_109382251.1 |
| | pyrophosphate synthase | NWU339 | |
| CycA01 | TR51_15710 cyclase | Kitasatospora griseola | KIQ65363.1 |
| CycB01 | terpene synthase family | Kitasatospora griseola | WP_043911627.1 |

In another embodiment, the bacterial cell is transformed with polynucleotides encoding acetyl-CoA C-acetyltransferase (AtoB) comprising the amino acid sequence of SEQ ID NO: 1, hydroxymethylglutaryl-CoA synthase (HMGS) comprising the amino acid sequence of SEQ ID NO:2, hydroxymethylglutaryl-CoA reductase (HMGR) comprising the amino acid sequence of SEQ ID NO:3, mevalonate kinase (MK) comprising the amino acid sequence of SEQ ID NO:4, phosphomevalonate kinase (PMK) comprising the amino acid sequence of SEQ ID NO:5, diphosphomevalonate decarboxylase (MVD) comprising the amino acid sequence of SEQ ID NO:6, Isopentenyl-diphosphate Delta-isomerase (IDI) comprising the amino acid sequence of SEQ ID NO:7, geranylgeranyl pyrophosphate synthase (GGPS) comprising the amino acid sequence of SEQ ID NO:14; TR51_15710 cyclase (CycA01) comprising the amino acid sequence of SEQ ID NO:18; and terpene synthase family (CycB01) comprising the amino acid sequence of SEQ ID NO:19.

TABLE 7
Encoded polypeptides

| Name | Encoded polypeptides | |
|---|---|---|
| CycA01 | KIQ65363.1 | |
| (SEQ ID | 1 | mpamsdadri aallkdraad pvtkfspspy |
| NO: 18) | | etgqflrise radvgtpqid yllatqrpdg |
| | 61 | lwgsvgfelv ptlgavagls srpeyadrag |
| | | vtdavarace klwelalgeg glpklpdtva |
| | 121 | seiivpslid llsevlqrhr pavggkagqs |
| | | qefpsppgak pelwrqlsdr iargqaipkt |
| | 181 | awhtleafhp lpkqfaatvt paadgavtcs |
| | | psstaawlsa vgtdagastr ayldeaqsry |
| | 241 | ggaipmgssm pyfevlwvln lvlkyfpdvp |
| | | ipreiieeia agfsdsgigg gpglppdgdd |
| | 301 | tayanlagdk lgapthpeil mkfwaedhfv |
| | | sypgeqtpse tvnahaleyl nhlrmrrgit |
| | 361 | efgavedaca ewvisqqted gcwydkwnvs |
| | | pyystaacve alldarkqde pqldslrrar |
| | 421 | ewllrhqtds ggwgmaepsp eetayavlal |
| | | dlfasrggeg aeecaaaisr akefftdesr |
| | 481 | enpplwmgkd lytpfrivdv tvmcgravvg ry |
| CycB01 | WP_043911627.1 | |
| (SEQ ID | 1 | mpdaiefehe grrnpnsaea esayssiiaa |
| NO: 19) | | ldlqesdyav isghsrivga aalvypdada |
| | 61 | etllaaslwt aclivnddrw dyvqedggrl |
| | | apgewfdgvt evvdtwrtag prlpdpffel |
| | 121 | vrttmsrlda algaeaadei gheikraita |
| | | mkwegvwney tkktslatyl sfrrgyctmd |
| | 181 | vqvvldkwin ggrsfaalrd dpvrraiddv |
| | | vvrfgclsnd yyswgrekka vdksnavril |
| | 241 | ndhagydest alahvrddcv qaitdldcie |
| | | esikrsghlg shaqelldyl achrpliyaa |
| | 301 | atwptetnry r |

In one embodiment, the polynucleotides may be codon-optimized for expression in the bacterial cell of interest. In other embodiments, the bacterial cell may be transfected with separate polynucleotides encoding each polypeptide, with a single polynucleotide encoding all of the polypeptides, or 2 or more polynucleotides each encoding 1 one or more of the polypeptides. In one embodiment, the polynucleotides are arranged in a single operon, with all polypeptides encoded by a single polynucleotide.

In one such embodiment, the polynucleotides encoding CycA01 and CycB01 are 5′ in the operon relative to polynucleotides encoding the other polypeptides having enzymatic activity. In another embodiment, the operon comprises, in 5′ to 3′ order, polynucleotides encoding CycA01, CycB01, atoB, HMGS, HMGR, MK, PMK, MVD, idi, and ggps.

In one embodiment, the operon comprises a heterologous promoter 5′ to the polynucleotides encoding the polypeptides. In one such embodiment, the operon comprises a heterologous promoter 5′ to the polynucleotide CycA01. Any heterologous promoter may be used as appropriate for an intended purpose. In various embodiments, the heterologous promoter comprises the nucleotide sequence of SEQ ID NO: 10, 21, 22, 24, or 25. In one embodiment, the heterologous promoter comprises the nucleotide sequence of SEQ ID NO: 10.

In another embodiment, the operon comprises a heterologous insulator 5′ to the polynucleotide sequence encoding CycA01. In a further embodiment, the heterologous insulator is 3′ to the heterologous promoter. Any heterologous insulator may be used as appropriate for an intended purpose. In some embodiments, the heterologous insulator comprises the nucleotide sequence of SEQ ID NO:11 or 23. In one embodiment, the heterologous insulator comprises the nucleotide sequence of SEQ ID NO:11.

In a further embodiment, the polynucleotides comprise a heterologous ribosome binding site (RBS) 5′ to all 10 of the genes encoding the polypeptides. Any heterologous RBS may be used as appropriate for an intended purpose. In one embodiment, the RBS comprises the nucleotide sequence of SEQ ID NO:12.

In another embodiment, the bacterial cell is transformed with the polynucleotide comprising the nucleotide sequence of SEQ ID NO:20, as shown below.

| SEQ ID NO: 20 | |
|---|---|
| LOCUS Kolavelool_Pathw 11521 bp DNA | |
| 1 | cggtggaaac gaggtcatca tttccttccg aaaaaacggt tgcatttaaa tcttacatat |
| 61 | gtaatacttt caaagactac atttgtaaga tttgaagctg tcaccggatg tgctttccgg |
| 121 | tctgatgagt ccgtgaggac gaaacagcct ctacaaataa ttttgtttaa ttaggaggat |
| 181 | gattatttat gccagctatg agtgatgcgg acagaattgc agcattgtta aaagatcgag |
| 241 | ctgcagatcc agtcacgaag ttttcacctt ctccatacga aacgggtcaa tttttgcgaa |
| 301 | tttcagaacg tgcagatgtg ggcacgcctc aaattgatta tcttttagca acacaacgtc |
| 361 | cagacggatt atggggtact gttggcttcg aattagttcc aactcttggt gctgttgctg |
| 421 | gtctttcttc aagacctgag tatgctgatc gtgctggtgt aacagacgca gttgctcgag |
| 481 | cttgtgaaaa gttatgggaa ttagcgttgg gcgaaggagg attaccaaaa ttgcctgata |
| 541 | cagttgcg=c agaaatcatt gtaccttctc ttattgactt gttaagtgaa gttttacaaa |
| 601 | gacatcgtcc agcagtggga ggtaaagcag gacaagaaca agagtttcca tcacctcctg |
| 661 | gagcaaaacc agagttatgg agacaattaa gtgatcgtat cgcacgtgga caagcaatcc |
| 721 | cgaaaactgc ttggcacaca ttagaagctt ttcatccatt acctaagcag tttgcagcta |
| 781 | ctgtaacacc agcagctgat ggtgctgtaa catgcagtcc ttcatctact gcagcgtggc |
| 841 | tatcagctgt tggtacagat gctggtgcgt ctacacgagc ttatttggat gaagcacaat |
| 901 | ctagatatgg tggtgcgatt cctatgggat caagtatgcc atattttgaa gtactatggg |
| 961 | tacttaatct agtcttaaaa tattttcctg atgttcctat acctagagaa ataattgaag |
| 1021 | aaattgctgc gggtttcagt gattctggta ttggtggtgg tccgggctta ccaccagatg |
| 1081 | gtgatgacac ggcatatgcg aacctagcag gagataaatt aggagcacct acacatccgg |
| 1141 | aaatattaat gaaattctgg gcggaagatc actttgttag ttatccaggt gaacaaacac |
| 1201 | catctgaaac tgtaaatgca catgcgctag aatatttaaa tcatcttcgt atgcgaagag |
| 1261 | gtataacaga attcggtgca gtggaagatg cgtgtgctga atgggtgata tcacaacaga |
| 1321 | ctgaggacgg ctgttggtac gataaatgga atgtctctcc ttactattca actgcagcat |
| 1381 | gtgtagaggc tttattagat gcacgtaaac aagatgaacc gcaattagac agtttacgtc |
| 1441 | gagctagaga gtggctttta agacatcaaa ctgatagtgg aggatggggt atggctgagc |
| 1501 | cgagtcctga agaaacagct tatgctgtac tagcgcttga tttatttgca agtagaggtg |
| 1561 | gcgaaggagc tgaagaatgt gcagcagcta tttcacgtgc aaaagaattt tttactgatg |
| 1621 | aaagtagaga aaatcctcca ttatggatgg gcaaagattt atatacacca tttcgtattg |
| 1681 | ttgatgtaac tgttatgtgt ggaagagcag tcgttggtcg ttattaatta ggaggatgat |
| 1741 | tatttatgcc agacgctata gaatttgagc atgaaggtag aagaaaccca aactcagctg |
| 1801 | aagcagaarc agcttatact tctattattg ctgcgttgga tttacaagaa tcagattatg |
| 1861 | ctgttatttc aggtcatact cgtattgtgg gcgctgcagc tttagtttat cctgatgctg |
| 1921 | atgcggaaac tttacttgct gcaagtttat ggacggcttg tttaattgtg aatgatgaca |
| 1981 | gatgggatta tgtccaagag gatggaggcc gtttagcacc tggtgaatgg ttcgatggtg |
| 2041 | taacggaagt agttgatact tggcgaacag ctggtccaag actacctgat ccattctttg |
| 2101 | aattggttcg aacaacaatg tctagattgg acgcggctct tggagcagag gcagcagatg |
| 2161 | aaataggtca cgaaattaaa cgagcgataa cagcgatgaa atgggaagga gtttggaatg |
| 2221 | aatatacgaa gaaaactagt ttagcgactt atttaagttt tcgtcgtgga tattgtacaa |
| 2281 | tggatgttca agtggtatta gataaatgga ttaatggtgg tagatctttt gctgctttac |
| 2341 | gagatgaccc ggtcagaaga gcaatcgatg acgttgtagt acgttttgga tgcttatcta |
| 2401 | atgattacta ctcatggggc cgtgaaaaga aagcagtaga taaaagtaat gcagttagaa |
| 2461 | tccttatgga tcatgcaggt tatgatgaat caactgcgtt ggctcatgtt cgtgacgatt |
| 2521 | gtgtacaagc aattactgat cttgattgta ttgaagaaag tataaaacgt tctggacacc |
| 2581 | taggtagtca tgcacaggag ttactagatt atttagcatg tcatcgtcca ttaatctacg |
| 2641 | cagcagcaac atggcctaca gaaacaaatc gttatagata attaggagga tgattattta |
| 2701 | tggctgaagc ttatattgtt gaagcagtta caacgcctgt cggtagaaga cgaggtggat |
| 2761 | tagctggcgt acacccagct gatttaggag cacatgcttt aacagctcta gttgcacgtt |
| 2821 | caggaattga tcctgcagcg gttgaagatg ttgtatttgg ttgcttagat acagttggtc |
| 2881 | cacaggctgg tgatattgca agaacttgtt ggttagcagc aggtcttcct gaagaagtac |
| 2941 | caggagtgac agtagataga caatgtggaa gttcacaaca agctgtgcat tttgctgcac |
| 3001 | aagctgttct atcaggtact caggatttgg tggtcgctgg aggagttcaa aatatgtcac |
| 3061 | aaattccaat agcatttgct tctagacaag cagctgaacc tttaggttta acgcaaggac |
| 3121 | cattcgcagg ttctgaaggt tggcgtgctc gatatggtga tcttccagtt aatcaatttc |
| 3181 | atggcgcaga actaattgcg gcaaagtggg gaattacacg acgtgatcaa gaagaatttg |
| 3241 | cattaagatc acatagaaga gcggtacgtg catctgacga aggtagattt gaccgagaaa |
| 3301 | cggttgcgta tggtaaagtc actgcggatg agggaccacg tagagataca agtttagaaa |
| 3361 | aaatggcggc tttagcacca gtaattgaag gtggaacgat tacagcagca tgttcaagtc |
| 3421 | aagtaagtga tggcgcagct gctatgttat tggcttcaga acgtgctttg agagaacatg |
| 3481 | gattaacgcc tagagcacgt gtgcatcatc taagtgtaag aggagaagat ccaattagaa |
| 3541 | tgttatctgc acctatccct gcaacagcac acgcattaaa aaaaactgct ttagcaattg |
| 3601 | atgatataga tttagtagaa atcaatgaag cgttcgcacc tgttgtactt gcatggttaa |
| 3661 | aagagactgg agcagatcct gaaaaagtga atgtcaatgg tggcgcaatc gctttaggac |
| 3721 | atcctttagg tgctacagct gttagattga tgacgacatt attacacgag ttagaaagaa |
| 3781 | ctggtggtcg ttttggttta caaacaatgt gtgaaggagg aggccaggcg aatgttacga |
| 3841 | tcatagaaag actttagtta ggaggatgat tatttatgac tataggtata gacaaaatag |
| 3901 | gtttcgctac atctcaatat gttttaaaac tagaagattt agcattggca cgtcaagtag |
| 3961 | atccagcaaa gttttctcaa ggtttattaa ttgaatcatt ttctgtagca cctatcacag |
| 4021 | aagatattat tacgttagca gcttctgcag cggaccaaat cctaactgat gaagatcgtg |
| 4081 | caaaaataga catggtaata ttagcaactg aaagtagtac tgaccaatca aaagcaagtg |
| 4141 | ctatttatgt tcaccatctt gtgggcattc aaccatttgc acgaagtttt gaagtaaagc |
| 4201 | aagcttgcza cagtgctaca gcagcattag attatgcaaa attacatgta gcaagtaaac |
| 4261 | cagatagtag agtattagtg atagcttcag acattgctcg ttatggagta ggatcaccag |
| 4321 | gagaaagtac acaaggaact ggtagtattg cattgttggt gacagcaaat ccacgtatct |
| 4381 | tagcattaaa taaagataat gttgctcaaa cacgtgatat aatggatttc tggcgaccaa |
| 4441 | attactcatt tacaccatac gttgatggta tttatagtac taaacaatat cttaattgct |
| 4501 | tagagactac atggcaagct tatcagaaac gagaaaattt acaattaagt gattttgctg |
| 4561 | cagtctgttt tcatatccct tttcctaaat tagcattaaa gggtttaaat aacattatgg |
| 4621 | ataatactgt ccctccagag catagagaaa aactaattga agcgttccaa gcatcaatta |
| 4681 | cttattctaa acaaattgcc aatatttata caggaagtct ttacttagga ttattgagtc |
| 4741 | ttttagaaaa tagtaaagtt ttacagtctg gtgacaagat cggttttttc tcatatggct |
| 4801 | caggagcagt ctctgaattt tattcaggcc aacttgttgc aggttatgat aaaatgttaa |
| 4861 | tgacaaatcg acaagcactt ttggatcaaa gaactagatt gtcagtttct aaatacgaag |
| 4921 | atttatttta tgaacaagtt caacttgatg ataatggtaa tgctaatttt gatatatatc |
| 4981 | taacaggtaa atttgctctt acagcaatca aagagcacca aagaatttac cacactaatg |
| 5041 | ataaaaatta attaggagga tgattattta tgactaaaac gaatttaaat tggtctggtt |
| 5101 | tctctaaaaa aacatttgaa gagcgtttac aattaataga aaaatttaaa ttactaaatg |
| 5161 | cagaaaactt aaatcaatta aaaacagatg ttttattacc tattcaaacg gcgaaccaga |
| 5221 | tgacagagaa tgtgttagct cgtttagcat tacctttttc aatagcgcca gactttttag |
| 5281 | ttaatggttc tacataccaa atgccatttg tcacagaaga gccttctgtg gtagctgcag |
| 5341 | caagttttgc tgcaaagtta ataaaaagaa gtggtggatt taaagcgcaa acacttaatc |
| 5401 | gtcaaatgat cggccaaatc gtgttatacg atattgatca aatcgataat gcgaaagcag |
| 5461 | ctatcttaca taaaacgaaa aaattgatag ctttggctaa caaagcatat ccatcaattg |
| 5521 | ttaaacgagg tggtggcgca agaactattc atttggaaga gaaaggtgaa ttcttaatat |
| 5581 | tttatttaac ggtagacaca caagaagcaa tgggtgctaa tatggtcaat acaatgatgg |
| 5641 | aagctttggt tccagactta actcgtttga gtaagggaca ttgtttaatg gcaatattat |
| 5701 | caaattatgc tacagaatca ctagttacaa catcttgcga aataccagtt agattattag |
| 5761 | atcgtgacaa aacaaaatct ttacaattag cacaaaaaat tgaattagca agtcgtctag |
| 5821 | ctcaagtgga tccttatcgt gcaacaacac ataataaagg tatttttaat ggtatagacg |
| 5881 | ctgtcgtcat tgctactgct aacgactggc gtgcaattga agcaggtgca catgcatatg |
| 5941 | cttcacgaaa tggtagttat caaggattaa gtcaatggca ttttgatcaa gacaaacagg |
| 6001 | ttttattagg tcaaatgaca ttacctatgc caattgcttc aaaaggaggt tcaataggct |
| 6061 | taaatcctac tgttagtatt gcacatgatt tattaaatca accagatgct aaaacactag |
| 6121 | ctcaacttat tgcaagtgta ggtttagcac aaaactttgc agcactaaaa gcgttaacta |
| 6181 | gttcaggtat ccaagcagct catatgaaat tacacgcgaa atctcttgct ttattggcag |
| 6241 | gcgctactca agatgaaatt gcaccattgg taaacgcttt gcttgctgat aaaccaatta |
| 6301 | atctagaaaa agctcacttt tatttatcac aactacgtca aagttgagtc gacctgcagg |
| 6361 | catgcaagct tggctgtttt ggttaggagg atgattattt atgaacgaga atataggtta |
| 6421 | tggaaaagca cattctaaaa ttattttaat aggtgaacat gcagtagtat atggctatcc |
| 6481 | agcaattgct cttcctctta cagatataga agtagtgtgt catatttttc cagcggataa |
| 6541 | accactagtt tttgacttct atgacacttt aagtacagca atatatgcgg cacttgatta |
| 6601 | tcttcaaaga ttgcaagaac caatagctta cgaaatagtt agtcaagtac cacagaagcg |
| 6661 | tggcatgggt agtagtgctg ctgttagtat tgcagcgatt agagctgttt tttcttattg |
| 6721 | tcaagagcct ttaagtgacg atttacttga aatcctagta aataaagcag aaataattgc |
| 6781 | acacacaaat ccttcaggat tagatgctaa aacatgctta tctgatcacg ctataaaatt |
| 6841 | tattagaaat ataggttttg agacaatcga aattgcttta aatggttatc taattattgc |
| 6901 | tgatacgggt attcatggtc atacaagaga agctgttaat aaagtcgctc agtttgaaga |
| 6961 | aacaaattta ccatatttag caaaacttgg tgcattaaca caagcattag aaagagctat |
| 7021 | caaccaaaaa aataaggtag caattggaca attaatgaca caagctcata gtgcattgaa |
| 7081 | agcaattggt gtatctatct ctaaagctga tcaattagtc gaagcagcat tacgagcagg |
| 7141 | tgcattaggt gctaaaatga caggtggtgg tctaggtggt tgtatgattg ctttagcaga |
| 7201 | tacaaaagat atggcagaaa aaatttcaca tagattaaag gaggaaggag cggtaaatac |
| 7261 | atggattcag atgttatagt taggaggatg attatttatg agtaactact gtgtacaaac |
| 7321 | gggcggtaaa ttatatttaa caggagaata tgcaatactt attccaggtc aaaaagcttt |
| 7381 | aattcactut attccattaa tgatgactgc tgaaatcagt ccagctgcac atattcaact |
| 7441 | tgcttcagat atgttttcac acaaagcggg tatgacacct gatgcaagtt atgctttaat |
| 7501 | tcaagcaaca gttaaaacat ttgcagatta cctaggacaa agtattgatc aattagaacc |
| 7561 | tttcagtctt attatcacgg gtaaaatgga acgtgatgga aaaaaattcg gtataggaag |
| 7621 | ttcaggatct gttactttat taactttaaa agcattgtca gcttattatc aaatcacatt |
| 7681 | aacaccagaa ttgttattta aattagctgc atatactttg ctaaaacagg gagataacgg |
| 7741 | ttctatggga gacatagcgt gcattgctta ccaaacatta gttgcatata catcttttga |
| 7801 | tcgtgaacaa gtgtcaaatt ggttacaaac tatgccatta aaaaaacttt tagtcaaaga |
| 7861 | ttggggatat catattcaag taattcagcc tgctttgcct tgtgatttct tagttggttg |
| 7921 | gactaagata cctgcaattt cacgtcaaat cattcagcaa gtaacggctt ctattacacc |
| 7981 | tgcgttttta cgtacgtcat atcaattaac acaatctgct atggtggctt tacaagaagg |
| 8041 | ccataaagaa gaattaaaaa aatcattggc tggagcttca cacttattaa aagaattaca |
| 8101 | ccctgctatt tatcatccta aattggttac attagttgct gcttgtcaaa aacaggatgc |
| 8161 | agtagcaaaa agtagtggaa gtggtggagg cgactgcggt attgctcttg catttaatca |
| 8221 | agatgcgcgt gatacattaa tttcaaaatg gcaagaagct gatatagctc ttttatacca |
| 8281 | agaaagatgg ggagagaatg actagttagg aggatgatta tttatggatc ctaatgtaat |
| 8341 | cacggttact tcatatgcaa atatagcaat aattaaatac tggggtaaag aaaatcaagc |
| 8401 | taaaatgatc ccttctacat cttctatttc attgacatta gaaaatatgt tcactactac |
| 8461 | ttcagtgtca tttttaccag acactgcaac ttcagatcaa ttttatataa acggtgtgtt |
| 8521 | acaaaatgat gaagaacata cgaaaatatc aacaattatt gatcaattcc gtcaaccagg |
| 8581 | acaagctttt gtaaaaatgg aaactcaaaa taacatgcct acagctgcag gattatcatc |
| 8641 | atcatcttca ggtttgtctg cattagtaaa ggcttgtgat caattgtttg atacacagct |
| 8701 | agatcaaaaa gcattagcac aaaaagcaaa atttgcgtct ggaagttcat cacgttcttt |
| 8761 | ctttggtcct gtcgcggcat gggataaaga ttctggcgca atatataaag ttgaaacgga |
| 8821 | cttgaaaatg gcgatgatta tgttagtttt aaacgcagca aaaaaaccta tttcatcacg |
| 8881 | tgaaggtatg aaattatgtc gtgatacatc aacaacattt gatgaatggg ttgaacagtc |
| 8941 | agcaattgac taccaacata tgttgacata tttaaaaaca aacaacttcg aaaaagttgg |
| 9001 | ccaattaaca gaagcgaatg cattggctat gcatgcaact acaaagacag cgaatcctcc |
| 9061 | ttttagttac ctaactaaag aatcttatca agctatggaa gctgtaaaag aattgcgtca |
| 9121 | agaaggttct gcatgctatt ttacaatgga tgcaggacct aatgtcaaag tactttgttt |
| 9181 | agagaaagat ttagcacaat tggcagagag attgggaaaa aattatcgaa ttatagtatc |
| 9241 | aaaaacaaaa gacttgccag acgtttaatt aggaggatga ttatttatga ctaatagaaa |
| 9301 | agatgatcat attaagtatg ctcttaaata ccaatcacct tataacgctt ttgatgatat |
| 9361 | tgaattaatt catcatagtc taccttctta tgatcttagt gacattgatt tatcaactca |
| 9421 | ctttgcagga caagattttg actttccatt ttatattaac gctatgacag gaggatctca |
| 9481 | aaaaggtaaa gctgttaatg aaaaattagc aaaagttgca gctgcaacag gtattgtaat |
| 9541 | ggtaacagga tcttatagtg cagctttgaa aaatccaaat gatgattctt atagattaca |
| 9601 | cgaggttgct gacaatctta aattagctac taacattggt ttagacaagc ctgttgcttt |
| 9661 | aggtcagcag acagtgcaag aaatgcaacc tttgtttctt caagtacatg ttaacgttat |
| 9721 | gcaagaat=g ttaatgccag aaggtgaaag agtttttcat acttggaaaa aacatctagc |
| 9781 | tgaatatgct agtcaaatac cagtcccagt aattcttaaa gaagtaggct ttggtatgga |
| 9841 | tgttaactct attaaattag cacatgattt gggtattcaa actttcgata taagtggtag |
| 9901 | aggtggaact agtttcgctt atatagaaaa tcaacgtggt ggcgatagat catacttgaa |
| 9961 | tgactggggt caaactacag ttcagtgtct tttaaatgca caaggcctaa tggatcaagt |
| 10021 | cgaaatatta gcatcaggtg gtgtacgtca tcctcttgat atgattaaat gttttgtttt |
| 10081 | aggcgctcgt gcagtaggat tatctagaac ggtgttggaa ttagttgaaa aatatccaac |
| 10141 | tgagagagta atcgctatag taaatggttg gaaggaagaa ttgaaaataa ttatgtgtgc |
| 10201 | attggattgc aaaacaataa aagagctaaa aggcgtggat tatttattat acggacgact |
| 10261 | tcaacaagta aattaagcga agacttaatc attaggagga tgattattta tggcaggaac |
| 10321 | ggcatgggat actgatatac caacgcttat ttcagtcaga gatgatgtgg atgcagtact |
| 10381 | tgaggactut gttgatgttc aagagagagc ggcacacgga ccagagatgg ctccattgtt |
| 10441 | cacaacggct agaagacttt tgtttagtaa cggtaaaaga ctaagaccat tattatgcgt |
| 10501 | ggcggggtgg caagcagctg gagctccagg tgacccgcat gctgtcttgc gtgttgcagc |
| 10561 | agcattagaa ctattccaga cattcgcgct aatacatgac gacgtaatgg atgacagtga |
| 10621 | tactagaaga ggtagaccta gtgcacatag agctttggca gcagagtaca tgagtggcgg |
| 10681 | tggacgatta agtaaggctg aagcgcatgg cagaggagct gccattttgt taggtgacct |
| 10741 | tttattagtc tggtctgacg aaatgcttgc tggtgcgggc cttgaggcga gagcacgagc |
| 10801 | tagagtactt ccattagttg actctatgag agctgagtta gtatacggtc agtacttaga |
| 10861 | tttattatct actgatagaa tgtcaggtga cgttgaagcg gcgctaagag tagcaagata |
| 10921 | taagacaggc aagtatacag ttgaaagacc acttcatttg ggtgttagtt tagcgggcgg |
| 10981 | tgacccacga ttattacaga cgtgtacgga ttttgcattg ccattaggtg aagcattcca |
| 11041 | acttcgtgat gatcttttaa acgtctttgg ggatccagtt cgtactggta agccggttgg |
| 11101 | tgacgatata cgtgaaggta aagctacagt attgttagct cttgctatac aggacgcgtc |
| 11161 | agaagctgac ttaaaattat taggtacttt agttggtagt cctgagttag gtagtgaaga |
| 11221 | tatagaccga ttcagaacag taatggagac tacaggtgca cgtcaacgtg tagaggacat |
| 11281 | gataatcgca cgtagagaca ctgcacttgc aactttggaa gaagcggatt taccacagga |
| 11341 | tgtagcagag acattaagac agatcgtttg gatggcgacg gagagacaat cataacggat |
| 11401 | gtaaacagag aaacgtcaca ctggctcacc ttcgggtggg cctttctgcg tttatagcct |
| 11461 | aatacacctg ttcgtggaga atataaaaag ccagattatt aatccggctt ttttattatt |
| 11521 | t |

In another aspect, the disclosure provides polynucleotides comprising the nucleotide sequence of any one of SEQ ID NO: 13, 17, and 20. In another embodiment, the disclosure provides recombinant expression vectors comprising the polynucleotide. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence is still considered “operably linked” to the coding sequence. Other such control sequences include, but are not limited to, polyadenylation signals, termination signals, etc. Such expression vectors can be of any type known in the art. In another embodiment, the present disclosure provides cells comprising the polynucleotide of the disclosure, such as a prokaryotic cell.

In a further embodiment, the disclosure provides compositions comprising a plurality of the engineered bacterial cell of any previous claim and a pharmaceutically acceptable carrier. The compositions may be of any suitable format for application of the compositions to the skin of a subject, including but not limited to a human subject. In one embodiment, the composition is formulated for topical application. In various non-limiting embodiments, the topical formulation may be in the form of a spray, a lotion, an oil, an ointment, a cream, a balm, a foam, a gel, or a salve.

The disclosure also provides methods to repel mosquitos, comprising contacting the skin of a subject with a plurality of the engineered bacterial cells of any embodiment or combination of embodiments herein, or with the composition of any embodiment or combination of embodiments herein, thereby repelling mosquitos from skin of the subject.

In another aspect, the disclosure provides methods to repel mosquitos, comprising contacting the skin of a subject with kaur-16-ene, kolavelool, or a combination thereof, thereby repelling mosquitos from skin of the subject.

In these methods, the subject may be any subject that can benefit from repelling mosquitos, including but not limited to human subjects. The compositions or compounds may be administered in any suitable formulation, including being formulated for topical application. In various non-limiting embodiments, the topical formulation may be in the form of a spray, a lotion, an oil, an ointment, a cream, a balm, a foam, a gel, or a salve

### Examples

### Engineering Human Skin Microbes to Produce Mosquito Repellent Terpenes.

Mosquitoes are a major source of vector-borne diseases including malaria, dengue, West Nile, zika and yellow fever, affecting millions every year. Vector-borne diseases account for 17% of all infectious diseases, resulting in >700,000 deaths annually (World Health Organization, 2022). Minimizing contact of mosquitoes is key to preventing the transmission of illnesses. Currently, there are several strategies for repelling mosquitoes: (i) physical barriers such as nets, (ii) Eliminating sites that are conducive for mosquito breeding, longevity and parasite sporogony such as ponds, swamps marshes and other bodies of still-water (iii) Pesticides and chemical sprays containing anti-mosquito compounds, of which the current gold standard, N, N-diethyl-meta-toluamide (DEET), can be found in over-the-counter sprays such as OFF!®. While DEET is a highly effective mosquito repellent, it requires consistent re-application due to its relatively short effective period and is cost prohibitive to much of the developing world.

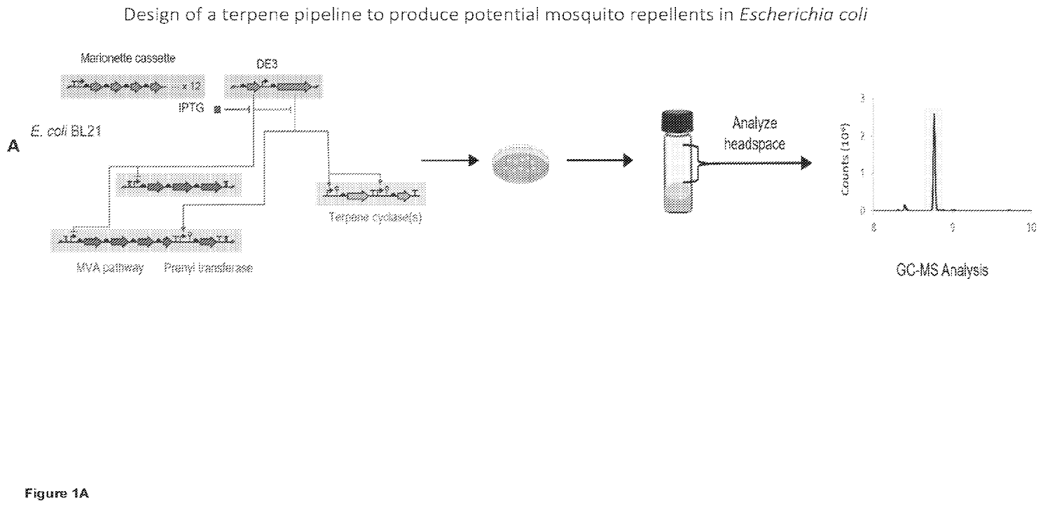

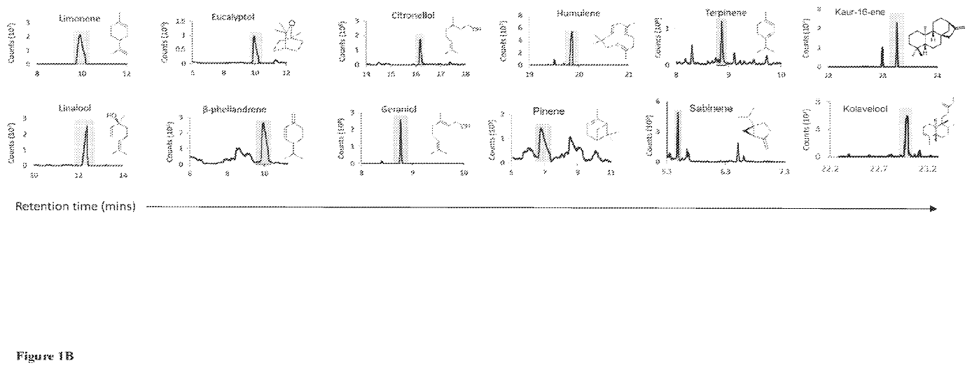

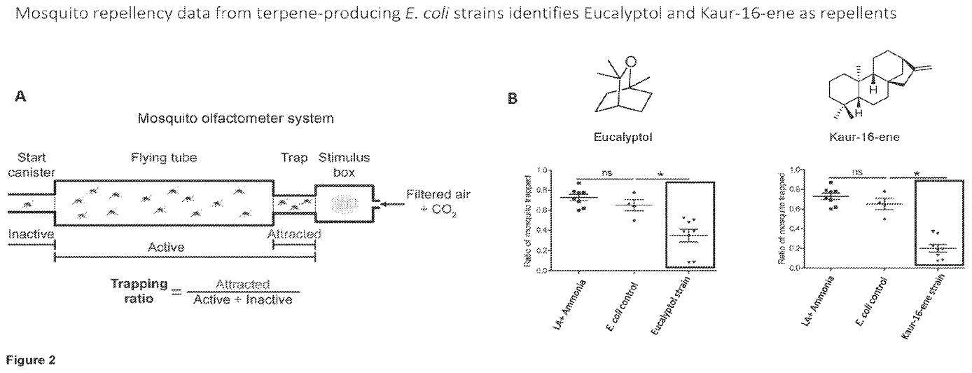

To mitigate disease transmission by mosquitoes, here we propose engineering bacteria on the surface of human skin to produce mosquito repellent terpenes. Terpenes are a structurally diverse class of natural products comprising of over 70,000 compounds. Terpenes are produced by plants, fungi and bacteria, act as pheromones and are involved in a number of important ecological functions such as plant cell signaling and defense. To screen for terpenes that are mosquito repellents, we first used a mosquito olfaction apparatus that can quantify the repellency of terpenes that are generated by Escherichia coli strains heterologously expressing a number of different terpene pathways (FIG. 1). The olfactometer used here is a dual-choice apparatus for screening the behavior of molecules that contribute to mosquito repulsion (FIG. 2A). This behavioral assay identified two terpenes as mosquito repellents: the monoterpene eucalyptol (also known as 1,8 cineole) and a diterpene repellent kaur-16-ene (FIG. 2B).

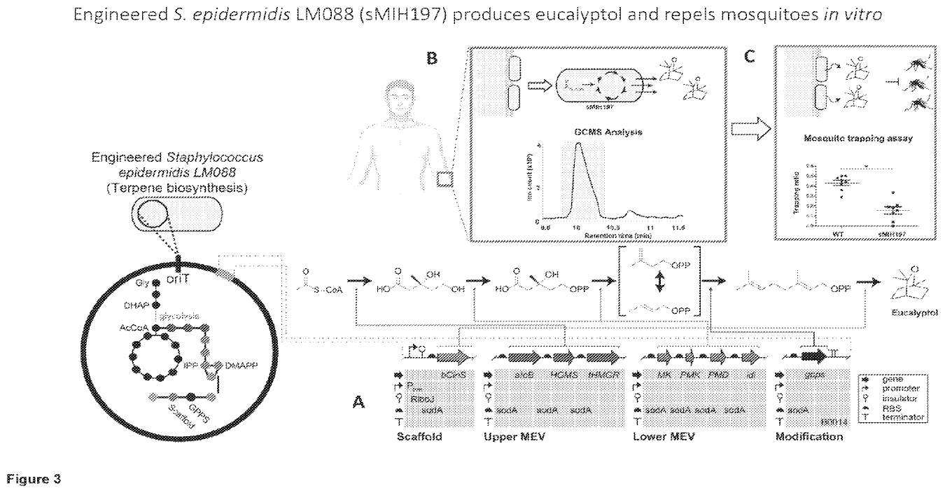

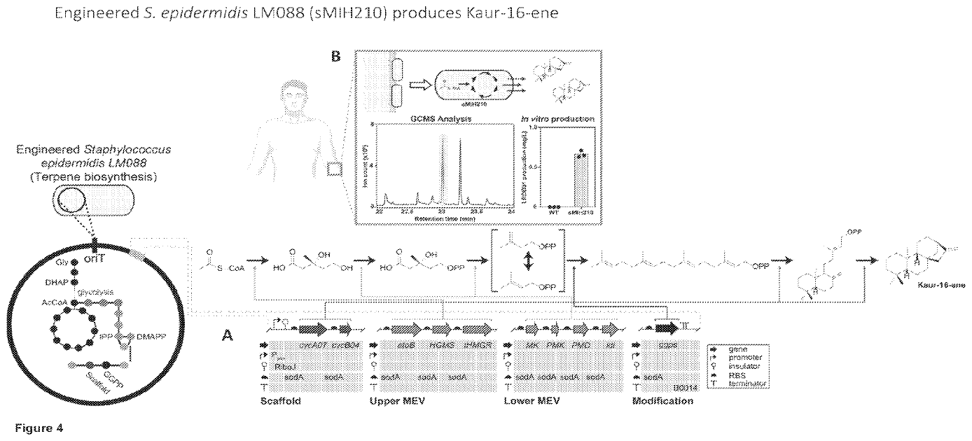

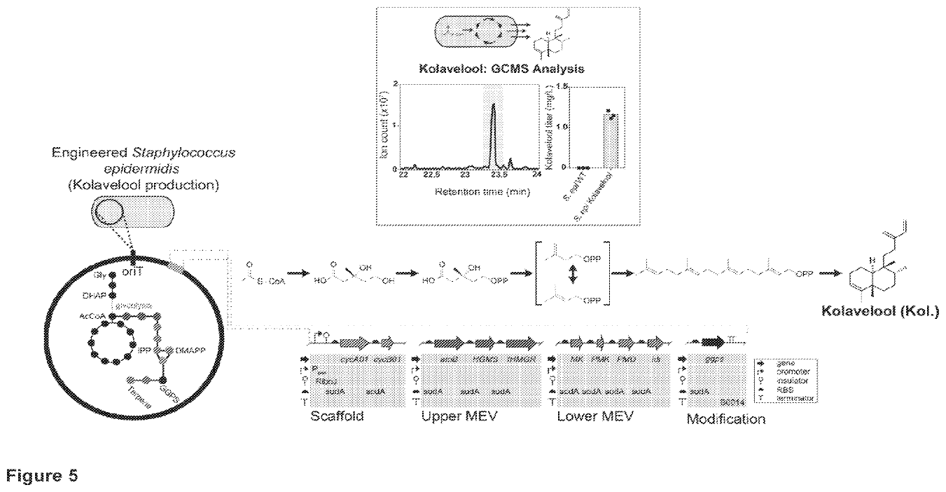

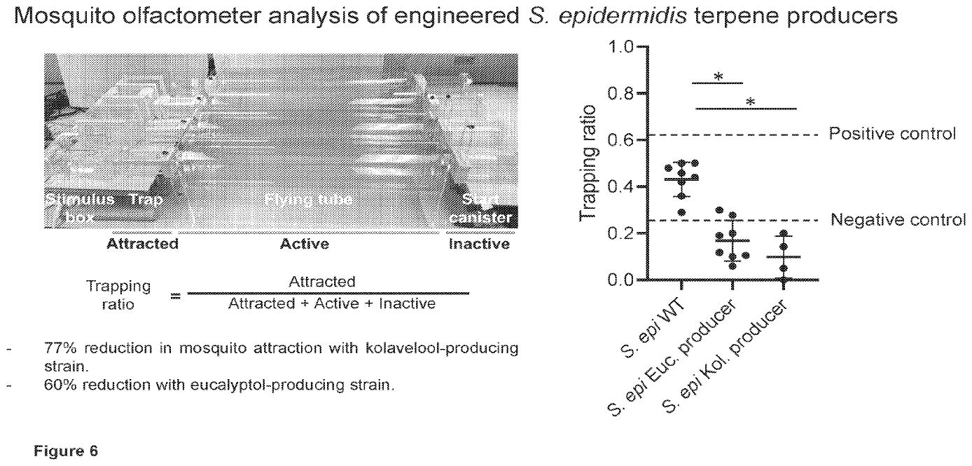

With this promising result, we moved forward with the complex task of integrating the biosynthetic pathways of eucalyptol and kaur-16-ene into the genome of a primary skin isolate, Staphylococcus epidermidis LM088. Manipulation of skin commensals most commonly found on humans has been particularly challenging, with only a handful of reported examples to date. These reported examples often involve introducing a single gene by plasmid transformation, which is not suitable for expressing & maintaining the multi-gene biosynthetic pathways required for terpene production. To address this challenge, we used a conjugation strategy optimized in the Voigt lab to integrate exogenous DNA in undomesticated bacteria (Voigt, Nat. Microbiol. 2018, 3, 9). This strategy, referred to as mini-ICE (integrative-conjugative elements), is able to integrate >100 Kb of DNA in undomesticated soil bacteria. Using this Bacillus subtilis-based conjugation strategy, the pathways of eucalyptol (sMIH197, S. epidermidis+eucalyptol, FIG. 3A) and kaur-16-ene (SMIH210, S. epidermidis+kaur-16-ene, FIG. 4A) were successfully integrated into the genome of an undomesticated primary skin isolate S. epidermidis LM088. Analysis of the engineered strains by solid-phase microextraction gas chromatography mass spectrometry (SPME-GC-MS) confirmed that sMIH197 and sMIH210 successfully produced eucalyptol (FIG. 3B) and kaur-16-ene (FIG. 4B), respectively. This is the first example of a primary skin isolate heterologously expressing terpene biosynthetic pathways and successfully generating terpenes of interest.

The mosquito olfactometer was then used to assess the efficacy of these engineered, terpene producing S. epidermidis strains as mosquito repellents. In vitro analysis of these engineered strains in an olfactometer showed that the eucalyptol-producing sMIH197 readily repels mosquitoes (FIG. 3C). This is the first demonstration of an engineered skin microbe successfully repelling mosquitoes. Ultimately, manipulating the skin microbiome to produce mosquito repellent terpenes such as eucalyptol and kaur-16-ene offers a novel strategy of repelling mosquitoes and minimizing transmission of mosquito associated illnesses.

Mini-ICEBs1 Conjugation of S. epidermidis LM088 to Generate Terpene Producing Skin Commensals

This B. subtilis-based conjugation strategy that was used in this disclosure was developed in the Voigt lab. More details can be found here: Voigt et al, Nat. Microbiol. 2018, 3, 9. The conjugation protocol was described in the 2018 manuscript with the following modifications for the integration of multi-gene terpene pathways in S. epidermidis:

Donor B. subtilis strains were generated by natural transformation of integrative plasmids. A single colony of B. subtilis was inoculated in 2 ml of LB+100 μg ml⁻¹ D-alanine and grown at 37° C. and 250 r.p.m. until optical density at 600 nm (OD600)=1. Cultures were then back-diluted 20-fold into minimal Davis (MD) medium (1× phosphatecitrate (PC) buffer (per liter: 10.7 g K₂HPO₄, 6 g KH₂PO₄, 1 g Na₃C₆H₅O₇, adjusted to pH 7.5 with KOH), 2% glucose, 3 mM MgSO₄, 0.02 mM C₆H₈FeNO₇, 14.5 mM C₄H₆KNO₄). 0.2 mL of the MD medium was mixed 0.02 mL of B. subtilis at OD600=1, 1000 ng of plasmid DNA (containing the full terpene pathway subcloned in to pJAB980), 50 μg ml⁻¹ tryptophan, 50 μg ml⁻¹ phenylalanine, and 100 μg ml⁻¹ D-alanine. Cells were grown for 24 h at 37° C. and 250 r.p.m and were plated onto LB selective medium containing 100 μg ml⁻¹ D-alanine and 5 μg ml⁻¹ of tetracycline. Successful B. subtilis colony donor strains were screened by PCR and used for conjugating primary skin commensals.

Donor (B. subtilis+terpene pathway) and recipient (S. epidermidis LM088) strains were picked from solid medium and inoculated in 2 mL of medium in 15 ml culture tube for 12 h at 37° C., 250 r.p.m. The dense overnight cultures were diluted 100-fold into 25 mL of medium in 250 mL Erlenmeyer flasks and grown at 37° C., 250 r.p.m. The recipient S. epidermidis LM088 strain was grown to an OD600 of 1. The donor B. subtilis strain was grown to OD600=0.35, after which 1% xylose (w/v) was added to the culture to induce ICE conjugation machinery and was further grown to an OD600=1, at which 1.5 mL of Donor and 1.5 mL of recipient were mixed and filtered onto sterile nitrocellulose membranes. Filters were placed on Petri dishes containing Tris-Spizzizen Salts (TSS) (per liter: 2 g NH₄Cl, 0.35 g K₂HPO₄·3H₂O, 6 g Tris base; pH to 7.5 with HCl) with 125 mM MgCl₂ and 2% agar. Matings were incubated for 5 h at 37° C. Cells were then rinsed off the filter using 3 ml TSS and plated on BHI+agar selective medium with 2.5 μg ml 1 of tetracycline. Successful S. epidermidis LM088 transconjugants were screened by colony PCR using primers specific to the terpene pathway of interest.

Details of Genes Used for the Production of Terpenes in S. epidermidis LM088

All genes related to terpene production were codon optimized using the codon usage shown in Table 8.

TABLE 8
Codon usage table
Staphylococcus epidermidis ATCC 12228 [gbbct]: 2485 CDS's (714252 codons)
fields: [triplet] [frequency: per thousand] ([number])

| | | | | | | | | | | | |
|---|---|---|---|---|---|---|---|---|---|---|---|
| UUU | 33.6 | (24014) | UCU | 15.9 | (11335) | UAU | 30.7 | (21944) | UGU | 5.2 | (3715) |
| UUC | 10.8 | (7718) | UCC | 2.6 | (1822) | UAC | 8.1 | (5787) | UGC | 1.1 | (797) |
| UUA | 51.0 | (36411) | UCA | 19.5 | (13932) | UAA | 2.5 | (1782) | UGA | 0.4 | (321) |
| UUG | 10.6 | (7544) | UCG | 3.1 | (2226) | UAG | 0.5 | (382) | UGG | 7.2 | (5129) |
| CUU | 15.0 | (10733) | CCU | 13.2 | (9445 | CAU | 19.7 | (14053) | CGU | 12.8 | (9147 |
| CUC | 4.0 | (2851) | CCC | 1.7 | (1238) | CAC | 5.1 | (3675) | CGC | 2.6 | (1880) |
| CUA | 9.4 | (6714) | CCA | 13.7 | (9750) | CAA | 37.0 | (26401) | CGA | 4.9 | (3503) |
| CUG | 2.1 | (1506) | CCG | 2.7 | (1946) | CAG | 5.1 | (3634) | CGG | 0.6 | (423) |
| AUU | 50.4 | (36020) | ACU | 19.6 | (14032) | AAU | 45.9 | (32800) | AGU | 17.6 | (12539) |
| AUC | 14.3 | (10205) | ACC | 3.6 | (2572) | AAC | 12.8 | (9138) | AGC | 4.5 | (3224) |
| AUA | 23.5 | (16776) | ACA | 25.8 | (18458) | AAA | 62.5 | (44648) | AGA | 13.4 | (9595) |
| AUG | 25.6 | (18286) | ACG | 6.9 | (4963) | AAG | 13.6 | (9747) | AGG | 1.4 | (1821) |
| GUU | 26.3 | (18756) | GCU | 22.2 | (15892) | GAU | 45.6 | (32547) | GGU | 30.2 | (21597) |
| GUC | 7.2 | (5137) | GCC | 4.5 | (3229) | GAC | 11.3 | (8063) | GGC | 7.3 | (5196) |
| GUA | 22.1 | (15763) | GCA | 25.2 | (18002) | GAA | 54.4 | (38863) | GGA | 17.4 | (12414) |
| GUG | 9.2 | (6559) | GCG | 6.5 | (4607) | GAG | 11.8 | (8452) | GGG | 4.8 | (3393) |

1. An engineered bacterial cell for producing a mosquito-repelling terpene, comprising an engineered terpene biosynthetic pathway introduced into a bacterial cell that is not otherwise capable of producing the terpene, wherein the engineered bacterial cell is capable of producing the mosquito-repelling terpene.

2. The engineered bacterial cell of claim 1, wherein the bacterial cell is a human skin microbe.

3. The engineered bacterial cell of claim 1, wherein the bacterial cell is Corynebacterium amycolatum, Staphylococcus caprae, or Staphylococcus epidermidis.

4-5. (canceled)

6. The engineered bacterial cell of claim 1, wherein the mosquito-repelling terpene is selected from the group consisting of eucalyptol, kaur-16-ene, and kolavelool.

7. The engineered bacterial cell of claim 1, wherein the mosquito-repelling terpene comprises eucalyptol.

8. The engineered bacterial cell of claim 7, wherein the bacterial cell is transformed with polynucleotides encoding the following polypeptides having enzymatic activity: acetyl-CoA C-acetyltransferase (AtoB), hydroxymethylglutaryl-CoA synthase (HMGS), hydroxymethylglutaryl-CoA reductase (HMGR), mevalonate kinase (MK), phosphomevalonate kinase (PMK), diphosphomevalonate decarboxylase (MVD), Isopentenyl-diphosphate Delta-isomerase (IDI), geranyldiphosphate synthase (GPPS), and 1,8 cineole synthase (bCinS).

9. The engineered bacterial cell of claim 8, wherein the bacterial cell is transformed with polynucleotides encoding acetyl-CoA C-acetyltransferase (AtoB) comprising the amino acid sequence of SEQ ID NO:1, hydroxymethylglutaryl-CoA synthase (HMGS) comprising the amino acid sequence of SEQ ID NO:2, hydroxymethylglutaryl-CoA reductase (HMGR) comprising the amino acid sequence of SEQ ID NO:3, mevalonate kinase (MK) comprising the amino acid sequence of SEQ ID NO:4, phosphomevalonate kinase (PMK) comprising the amino acid sequence of SEQ ID NO:5, diphosphomevalonate decarboxylase (MVD) comprising the amino acid sequence of SEQ ID NO:6, Isopentenyl-diphosphate Delta-isomerase (IDI) comprising the amino acid sequence of SEQ ID NO:7, geranyldiphosphate synthase (GPPS) comprising the amino acid sequence of SEQ ID NO:8, and 1,8 cineole synthase (bCinS) comprising the amino acid sequence of SEQ ID NO:9.

10. The engineered bacterial cell of claim 8, wherein the polynucleotides are codon-optimized for expression in the bacterial cell.

11. The engineered bacterial cell of claim 8, wherein the polynucleotides are arranged in a single operon.

12. The engineered bacterial cell of claim 11, wherein the polynucleotide encoding bCinS is 5′ in the operon relative to polynucleotides encoding the other polypeptides having enzymatic activity.

13. The engineered bacterial cell of claim 11, wherein the operon comprises, in 5′ to 3′ order, polynucleotides encoding bCinS, atoB, HMGS, HMGR, MK, PMK, MVD, idi, and gpps.

14. The engineered bacterial cell of claim 11, wherein the operon comprises a heterologous promoter 5′ to the polynucleotide sequence encoding bCinS.

15. The engineered bacterial cell of claim 14, wherein the heterologous promoter comprises the nucleotide sequence of SEQ ID NO: 10, 21, 22, 24, o 25.

16-21. (canceled)

22. The engineered bacterial cell of claim 1, wherein the mosquito-repelling terpene comprises kaur-16-ene, wherein the bacterial cell is transformed with polynucleotides encoding the following polypeptides having enzymatic activity: wherein the bacterial cell is transformed with polynucleotides encoding the following polypeptides having enzymatic activity: acetyl-CoA C-acetyltransferase (AtoB), hydroxymethylglutaryl-CoA synthase (HMGS), hydroxymethylglutaryl-CoA reductase (HMGR), mevalonate kinase (MK), phosphomevalonate kinase (PMK), diphosphomevalonate decarboxylase (MVD), Isopentenyl-diphosphate Delta-isomerase (IDI), geranylgeranyl pyrophosphate synthase (GGPS), PtmT2 cyclase (CycA07), and Hypothetical cyclase (CycB04).

23-36. (canceled)

37. The engineered bacterial cell of claim 1, wherein the mosquito-repelling terpene comprises kolavelool, wherein the bacterial cell is transformed with polynucleotides encoding the following polypeptides having enzymatic activity: acetyl-CoA C-acetyltransferase (AtoB), hydroxymethylglutaryl-CoA synthase (HMGS), hydroxymethylglutaryl-CoA reductase (HMGR), mevalonate kinase (MK), phosphomevalonate kinase (PMK), diphosphomevalonate decarboxylase (MVD), Isopentenyl-diphosphate Delta-isomerase (IDI), geranylgeranyl pyrophosphate synthase (GGPS); TR51_15710 cyclase (CycA01); and terpene synthase family (CycB01).

38-51. (canceled)

52. A polynucleotide comprising the nucleotide sequence of any one of SEQ ID NO: 13, 17, and 20.

53. A recombinant expression vector comprising the polynucleotide of claim 52.

54. A recombinant host cell comprising the recombinant expression vector of claim 53.

55. A composition comprising a plurality of the engineered bacterial cell of claim 1 and a pharmaceutically acceptable carrier.

56. (canceled)

57. A method to repel mosquitos from a subject, comprising contacting the skin of the subject with (a) a plurality of the engineered bacterial cells of claim 1, or (b) kaur-16-ene, kolavelool, or a combination thereof, thereby repelling mosquitos from skin of the subject.

58-59. (canceled)


SEQUENCE LISTING (25 sequences)

SEQ ID NO: 1 - protein, 385 aa, synthetic construct
MAEAYIVEAVRTPVGRRRGGLAGVHPADLGAHALTALVARSGIDPAAVEDVVFGCLDTVGPQAGDIARTCWLAAGLPEEVPGVTVDRQCGSSQQAVHFAAQAVLSGTQDLVVAGGVQNMSQIPIAFASRQAAEPLGLTQGPFAGSEGWRARYGDLPVNQFHGAELIAAKWGITRRDQEEFALRSHRRAVRASDEGRFDRETVAYGKVTADEGPRRDTSLEKMAALAPVIEGGTITAACSSQVSDGAAAMLLASERALREHGLTPRARVHHLSVRGEDPIRMLSAPIPATAHALKKTALAIDDIDLVEINEAFAPVVLAWLKETGADPEKVNVNGGAIALGHPLGATGVRLMTTLLHELERTGGRFGLQTMCEGGGQANVTIIERL

SEQ ID NO: 2 - protein, 404 aa, synthetic construct
MGKTIKITKRRTNMTIGIDKIGFATSQYVLKLEDLALARQVDPAKFSQGLLIESFSVAPITEDIITLAASAADQILTDEDRAKIDMVILATESSTDQSKASAIYVHHLVGIQPFARSFEVKQACYSATAALDYAKLHVASKPDSRVLVIASDIARYGVGSPGESTQGSGSIALLVTANPRILALNKDNVAQTRDIMDFWRPNYSFTPYVDGIYSTKQYLNCLETTWQAYQKRENLQLSDFAAVCFHIPFPKLALKGLNNIMDNTVPPEHREKLIEAFQASITYSKQIGNIYTGSLYLGLLSLLENSKVLQSGDKIGFFSYGSGAVSEFYSGQLVAGYDKMLMTNRQALLDQRTRLSVSKYEDLFYEQVQLDDNGNANFDIYLTGKFALTAIKEHQRIYHTNDKN

SEQ ID NO: 3 - protein, 425 aa, synthetic construct
MTKTNLNWSGFSKKTFEERLQLIEKFKLLNAENLNQLKTDVLLPIQTANQMTENVLGRLALPFSIAPDFLVNGSTYQMPFVTEEPSVVAAASFAAKLIKRSGGFKAQTLNRQMIGQIVLYDIDQIDNAKAAILHKTKKLIALANKAYPSIVKRGGGARTIHLEEKGEFLIFYLTVDTQEAMGANMVNTMMEALVPDLTRLSKGHCLMAILSNYATESLVTTSCEIPVRLLDRDKTKSLQLAQKIELASRLAQVDPYRATTHNKGIFNGIDAVVIATGNDWRAIEAGAHAYASRNGSYQGLSQWHFDQDKQVLLGQMTLPMPIASKGGSIGLNPTVSIAHDLLNQPDAKTLAQLIASVGLAQNFAALKALTSSGIQAGHMKLHAKSLALLAGATQDEIAPLVNALLADKPINLEKAHFYLSQLRQS

SEQ ID NO: 4 - protein, 292 aa, synthetic construct
MNENIGYGKAHSKIILIGEHAVVYGYPAIALPLTDIEVVCHIFPADKPLVFDFYDTLSTAIYAALDYLQRLQEPIAYEIVSQVPQKRGMGSSAAVSIAAIRAVFSYCQEPLSDDLLEILVNKAEIIAHTNPSGLDAKTCLSDHAIKFIRNIGFETIEIALNGYLIIADTGIHGHTREAVNKVAQFEETNLPYLAKLGALTQALERAINQKNKVAIGQLMTQAHSALKAIGVSISKADQLVEAALRAGALGAKMTGGGLGGCMIALADTKDMAEKISHRLKEEGAVNTWIQML

SEQ ID NO: 5 - protein, 351 aa, synthetic construct
MAKTTVLSFQKQRIYQMSNYCVQTGGKLYLTGEYAILIPGQKALIHFIPLMMTAEISPAAHIQLASDMFSHKAGMTPDASYALIQATVKTFADYLGQSIDQLEPFSLIITGKMERDGKKFGIGSSGSVTLLTLKALSAYYQITLTPELLFKLAAYTLLKQGDNGSMGDIACIAYQTLVAYTSFDREQVSNWLQTMPLKKLLVKDWGYHIQVIQPALPCDFLVGWTKIPAISRQMIQQVTASITPAFLRTSYQLTQSAMVALQEGHKEELKKSLAGASHLLKELHPAIYHPKLVTLVAACQKQDAVAKSSGSGGGDCGIALAFNQDARDTLISKWQEADIALLYQERWGEND

SEQ ID NO: 6 - protein, 314 aa, synthetic construct
MDPNVITVTSYANIAIIKYWGKENQAKMIPSTSSISLTLENMFTTTSVSFLPDTATSDQFYINGVLQNDEEHTKISTIIDQFRQPGQAFVKMETQNNMPTAAGLSSSSSGLSALVKACDQLFDTQLDQKALAQKAKFASGSSSRSFFGPVAAWDKDSGAIYKVETDLKMAMIMLVLNAAKKPISSREGMKLCRDTSTTFDEWVEQSAIDYQHMLTYLKTNNFEKVGQLTEANALAMHATTKTANPPFSYLTKESYQAMEAVKELRQEGFACYFTMDAGPNVKVLCLEKDLAQLAERLGKNYRIIVSKTKDLPDV

SEQ ID NO: 7 - protein, 329 aa, synthetic construct
MTNRKDDHIKYALKYQSPYNAFDDIELIHHSLPSYDLSDIDLSTHFAGQDFDFPFYINAMTGGSQKGKAVNEKLAKVAAATGIVMVTGSYSAALKNPNDDSYRLHEVADNLKLATNIGLDKPVALGQQTVQEMQPLFLQVHVNVMQELLMPEGERVFHTWKKHLAEYASQIPVPVILKEVGFGMDVNSIKLAHDLGIQTFDISGRGGTSFAYIENQRGGDRSYLNDWGQTTVQCLLNAQGLMDQVEILASGGVRHPLDMIKCFVLGARAVGLSRTVLELVEKYPTERVIAIVNGWKEELKIIMCALDCKTIKELKGVDYLLYGRLQQVN

SEQ ID NO: 8 - protein, 381 aa, synthetic construct
MAYSAMATMGYNGMAASCHTLHPTSPLKPFHGASTSLEAFNGEHMGLLRGYSKRKLSSYKNPASRSSNATVAQLLNPPQKGKKAVEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 9 - protein, 330 aa, synthetic construct
MPAGHEEFDIPFPSRVNPFHARAEDRHVAWMRAMGLITGDAAEATYRRWSPAKVGARWFYLAQGEDLDLGCDIFGWFFAYDDHFDGPTGTDPRQTAAFVNRTVAMLDPRADPTGEHPLNIAFHDLWQRESAPMSPLWQRRAVDHWTQYLTAHITEATNRTRHTSPTIADYLELRHRTGFMPPLLDLIERVWRAEIPAPVYTTPEVQTLLHTTNQNINIVNDVLSLEKEEAHGDPHNLVLVIQHERQSTRQQALATARRMIDEWTDTFIRTEPRLPALCGRLGIPLADRTSLYTAVEGMRAAIRGNYDWCAETNRYAVHRPTGTGRATTPW

SEQ ID NO: 10 - DNA, 95 bp, synthetic construct
  source 1..95: mol_type = other DNA
cggtggaaacgaggtcatcatttccttccgaaaaaacggttgcatttaaatcttacatatgtaatactttcaaagactacatttgtaagatttga

SEQ ID NO: 11 - DNA, 95 bp, synthetic construct; identical to SEQ ID NO: 10
  source 1..95: mol_type = other DNA

SEQ ID NO: 12 - DNA, 18 bp, synthetic construct
  source 1..18: mol_type = other DNA
ttaggaggatgattattt

SEQ ID NO: 13 - DNA, 10078 bp, synthetic construct
  source 1..10078: mol_type = other DNA
cggtggaaacgaggtcatcatttccttccgaaaaaacggttgcatttaaatcttacatatgtaatactttcaaagactacatttgtaagatttgaagctgtcaccggatgtgctttccggtctgatgagtccgtgaggacgaaacagcctctacaaataattttgtttaattaggaggatgattatttatgcctgcaggtcatgaagaattcgatataccatttccttcacgtgtaaatccatttcatgcacgagcagaagatcgtcatgttgcttggatgagagctatgggtttaattacgggtgatgctgctgaagcgacttatcgtcgttggtcaccggctaaagtaggtgcaagatggttttatcttgctcaaggtgaagatttagatttgggttgtgacatttttggatggtttttcgcttatgatgaccattttgatggtccgactggcactgatccaagacaaactgcagcttttgttaatagaactgtcgcaatgttagatcctcgtgcagatcctacaggtgaacatccattaaatattgcatttcatgatctatggcaaagagagagtgcacctatgagtccattatggcaacgacgtgctgttgatcactggacacaatatttaacagctcatattactgaagcaactaatcgtacaagacatacatcacctacaattgcagattacttagaattacgacatagaactggatttatgccgccattgcttgacttaattgaaagagtatggcgtgctgaaatacctgcgcctgtatatacgacaccagaggtgcaaacattgctacacacaacaaatcagaatatcaacatagttaatgacgttttatctcttgagaaagaggaagcgcacggcgatccacataacctagtattagtgattcaacatgaaagacaaagtactagacaacaagcattagcaactgcacgaagaatgattgatgaatggactgatacattcatccgtactgaaccaagacttccagcattatgcggacgattaggaataccacttgctgatcgtacatctttatacacagctgtcgaaggtatgcgtgctgcaattagaggaaattatgattggtgtgctgaaacaaatcgttatgctgttcatcgtcctacaggaacgggtcgtgcgacgactccttggtaattaggaggatgattatttatggctgaagcttatattgttgaagcagttagaacgcctgtcggtagaagacgaggtggattagctggcgtacacccagctgatttaggagcacatgctttaacagctctagttgcacgttcaggaattgatcctgcagcggttgaagatgttgtatttggttgcttagatacagttggtccacaggctggtgatattgcaagaacttgttggttagcagcaggtcttcctgaagaagtaccaggagtgacagtagatagacaatgtggaagttcacaacaagctgtgcattttgctgcacaagctgttctatcaggtactcaggatttggtggtcgctggaggagttcaaaatatgtcacaaattccaatagcatttgcttctagacaagcagctgaacctttaggtttaacgcaaggaccattcgcaggttctgaaggttggcgtgctcgatatggtgatcttccagttaatcaatttcatggcgcagaactaattgcggcaaagtggggaattacacgacgtgatcaagaagaatttgcattaagatcacatagaagagcggtacgtgcatctgacgaaggtagatttgaccgagaaacggttgcgtatggtaaagtcactgcggatgagggaccacgtagagatacaagtttagaaaaaatggcggctttagcaccagtaattgaaggtggaacgattacagcagcatgttcaagtcaagtaagtgatggcgcagctgctatgttattggcttcagaacgtgctttgagagaacatggattaacgcctagagcacgtgtgcatcatctaagtgtaagaggagaagatccaattagaatgttatctgcacctatccctgcaacagcacacgcattaaaaaaaactgctttagcaattgatgatatagatttagtagaaatcaatgaagcgttcgcacctgttgtacttgcatggttaaaagagactggagcagatcctgaaaaagtgaatgtcaatggtggcgcaatcgctttaggacatcctttaggtgctacaggtgttagattgatgacgacattattacacgagttagaaagaactggtggtcgttttggtttacaaacaatgtgtgaaggaggaggccaggcgaatgttacgatcatagaaagactttagttaggaggatgattatttatgactataggtatagacaaaataggtttcgctacatctcaatatgttttaaaactagaagatttagcattggcacgtcaagtagatccagcaaagttttctcaaggtttattaattgaatcattttctgtagcacctatcacagaagatattattacgttagcagcttctgcagcggaccaaatcctaactgatgaagatcgtgcaaaaatagacatggtaatattagcaactgaaagtagtactgaccaatcaaaagcaagtgctatttatgttcaccatcttgtgggcattcaaccatttgcacgaagttttgaagtaaagcaagcttgctacagtgctacagcagcattagattatgcaaaattacatgtagcaagtaaaccagatagtagagtattagtgatagcttcagacattgctcgttatggagtaggatcaccaggagaaagtacacaaggaagtggtagtattgcattgttggtgacagcaaatccacgtatcttagcattaaataaagataatgttgctcaaacacgtgatataatggatttctggcgaccaaattactcatttacaccatacgttgatggtatttatagtactaaacaatatcttaattgcttagagactacatggcaagcttatcagaaacgagaaaatttacaattaagtgattttgctgcagtctgttttcatatcccttttcctaaattagcattaaagggtttaaataacattatggataatactgtccctccagagcatagagaaaaactaattgaagcgttccaagcatcaattacttattctaaacaaattggcaatatttatacaggaagtctttacttaggattattgagtcttttagaaaatagtaaagttttacagtctggtgacaagatcggttttttctcatatggctcaggagcagtctctgaattttattcaggccaacttgttgcaggttatgataaaatgttaatgacaaatcgacaagcacttttggatcaaagaactagattgtcagtttctaaatacgaagatttattttatgaacaagttcaacttgatgataatggtaatgctaattttgatatatatctaacaggtaaatttgctcttacagcaatcaaagagcaccaaagaatttaccacactaatgataaaaattaattaggaggatgattatttatgactaaaacgaatttaaattggtctggtttctctaaaaaaacatttgaagagcgtttacaattaatagaaaaatttaaattactaaatgcagaaaacttaaatcaattaaaaacagatgttttattacctattcaaacggcgaaccagatgacagagaatgtgttaggtcgtttagcattacctttttcaatagcgccagactttttagttaatggttctacataccaaatgccatttgtcacagaagagccttctgtggtagctgcagcaagttttgctgcaaagttaataaaaagaagtggtggatttaaagcgcaaacacttaatcgtcaaatgatcggccaaatcgtgttatacgatattgatcaaatcgataatgcgaaagcagctatcttacataaaacgaaaaaattgatagctttggctaacaaagcatatccatcaattgttaaacgaggtggtggcgcaagaactattcatttggaagagaaaggtgaattcttaatattttatttaacggtagacacacaagaagcaatgggtgctaatatggtcaatacaatgatggaagctttggttccagacttaactcgtttgagtaagggacattgtttaatggcaatattatcaaattatgctacagaatcactagttacaacatcttgcgaaataccagttagattattagatcgtgacaaaacaaaatctttacaattagcacaaaaaattgaattagcaagtcgtctagctcaagtggatccttatcgtgcaacaacacataataaaggtatttttaatggtatagacgctgtcgtcattgctactggtaacgactggcgtgcaattgaagcaggtgcacatgcatatgcttcacgaaatggtagttatcaaggcttaagtcaatggcattttgatcaagacaaacaggttttattaggtcaaatgacattacctatgccaattgcttcaaaaggaggttcaataggcttaaatcctactgttagtattgcacatgatttattaaatcaaccagatgctaaaacactagctcaacttattgcaagtgtaggtttagcacaaaactttgcagcactaaaagcgttaactagttcaggtatccaagcaggtcatatgaaattacacgcgaaatctcttgctttattggcaggcgctactcaagatgaaattgcaccattggtaaacgctttgcttgctgataaaccaattaatctagaaaaagctcacttttatttatcacaactacgtcaaagttgagtcgacctgcaggcatgcaagcttggctgttttggttaggaggatgattatttatgaacgagaatataggttatggaaaagcacattctaaaattattttaataggtgaacatgcagtagtatatggctatccagcaattgctcttcctcttacagatatagaagtagtgtgtcatatttttccagcggataaaccactagtttttgacttctatgacactttaagtacagcaatatatgcggcacttgattatcttcaaagattgcaagaaccaatagcttacgaaatagttagtcaagtaccacagaagcgtggcatgggtagtagtgctgctgttagtattgcagcgattagagctgttttttcttattgtcaagagcctttaagtgacgatttacttgaaatcctagtaaataaagcagaaataattgcacacacaaatccttcaggattagatgctaaaacatgcttatctgatcacgctataaaatttattagaaatataggttttgagacaatcgaaattgctttaaatggttatctaattattgctgatacgggtattcatggtcatacaagagaagctgttaataaagtcgctcagtttgaagaaacaaatttaccatatttagcaaaacttggtgcattaacacaagcattagaaagagctatcaaccaaaaaaataaggtagcaattggacaattaatgacacaagctcatagtgcattgaaagcaattggtgtatctatctctaaagctgatcaattagtcgaagcagcattacgagcaggtgcattaggtgctaaaatgacaggtggtggtctaggtggttgtatgattgctttagcagatacaaaagatatggcagaaaaaatttcacatagattaaaggaggaaggagcggtaaatacatggattcagatgttatagttaggaggatgattatttatgagtaactactgtgtacaaacgggcggtaaattatatttaacaggagaatatgcaatacttattccaggtcaaaaagctttaattcactttattccattaatgatgactgctgaaatcagtccagctgcacatattcaacttgcttcagatatgttttcacacaaagcgggtatgacacctgatgcaagttatgctttaattcaagcaacagttaaaacatttgcagattacctaggacaaagtattgatcaattagaacctttcagtcttattatcacgggtaaaatggaacgtgatggaaaaaaattcggtataggaagttcaggatctgttactttattaactttaaaagcattgtcagcttattatcaaatcacattaacaccagaattgttatttaaattagctgcatatactttgctaaaacagggagataacggttctatgggagacatagcgtgcattgcttaccaaacattagttgcatatacatcttttgatcgtgaacaagtgtcaaattggttacaaactatgccattaaaaaaacttttagtcaaagattggggatatcatattcaagtaattcagcctgctttgccttgtgatttcttagttggttggactaagatacctgcaatttcacgtcaaatgattcagcaagtaacggcttctattacacctgcgtttttacgtacgtcatatcaattaacacaatctgctatggtggctttacaagaaggccataaagaagaattaaaaaaatcattggctggagcttcacacttattaaaagaattacaccctgctatttatcatcctaaattggttacattagttgctgcttgtcaaaaacaggatgcagtagcaaaaagtagtggaagtggtggaggcgactgcggtattgctcttgcatttaatcaagatgcgcgtgatacattaatttcaaaatggcaagaagctgatatagctcttttataccaagaaagatggggagagaatgactagttaggaggatgattatttatggatcctaatgtaatcacggttacttcatatgcaaatatagcaataattaaatactggggtaaagaaaatcaagctaaaatgatcccttctacatcttctatttcattgacattagaaaatatgttcactactacttcagtgtcatttttaccagacactgcaacttcagatcaattttatataaacggtgtgttacaaaatgatgaagaacatacgaaaatatcaacaattattgatcaattccgtcaaccaggacaagcttttgtaaaaatggaaactcaaaataacatgcctacagctgcaggattatcatcatcatcttcaggtttgtctgcattagtaaaggcttgtgatcaattgtttgatacacagctagatcaaaaagcattagcacaaaaagcaaaatttgcgtctggaagttcatcacgttctttctttggtcctgtcgcggcatgggataaagattctggcgcaatatataaagttgaaacggacttgaaaatggcgatgattatgttagttttaaacgcagcaaaaaaacctatttcatcacgtgaaggtatgaaattatgtcgtgatacatcaacaacatttgatgaatgggttgaacagtcagcaattgactaccaacatatgttgacatatttaaaaacaaacaacttcgaaaaagttggccaattaacagaagcgaatgcattggctatgcatgcaactacaaagacagcgaatcctccttttagttacctaactaaagaatcttatcaagctatggaagctgtaaaagaattgcgtcaagaaggttttgcatgctattttacaatggatgcaggacctaatgtcaaagtactttgtttagagaaagatttagcacaattggcagagagattgggaaaaaattatcgaattatagtatcaaaaacaaaagacttgccagacgtttaattaggaggatgattatttatgactaatagaaaagatgatcatattaagtatgctcttaaataccaatcaccttataacgcttttgatgatattgaattaattcatcatagtctaccttcttatgatcttagtgacattgatttatcaactcactttgcaggacaagattttgactttccattttatattaacgctatgacaggaggatctcaaaaaggtaaagctgttaatgaaaaattagcaaaagttgcagctgcaacaggtattgtaatggtaacaggatcttatagtgcagctttgaaaaatccaaatgatgattcttatagattacacgaggttgctgacaatcttaaattagctactaacattggtttagacaagcctgttgctttaggtcagcagacagtgcaagaaatgcaacctttgtttcttcaagtacatgttaacgttatgcaagaattgttaatgccagaaggtgaaagagtttttcatacttggaaaaaacatctagctgaatatgctagtcaaataccagtcccagtaattcttaaagaagtaggctttggtatggatgttaactctattaaattagcacatgatttgggtattcaaactttcgatataagtggtagaggtggaactagtttcgcttatatagaaaatcaacgtggtggcgatagatcatacttgaatgactggggtcaaactacagttcagtgtcttttaaatgcacaaggcctaatggatcaagtcgaaatattagcatcaggtggtgtacgtcatcctcttgatatgattaaatgttttgttttaggcgctcgtgcagtaggattatctagaacggtgttggaattagttgaaaaatatccaactgagagagtaatcgctatagtaaatggttggaaggaagaattgaaaataattatgtgtgcattggattgcaaaacaataaaagagctaaaaggcgtggattatttattatacggacgacttcaacaagtaaattaagcgaagacttaatcattaggaggatgattatttatggcttattcagcaatggctactatgggttataacggtatggctgcatcatgtcatacacttcatccaacatctccattaaaaccttttcacggtgcatcaacttcacttgaagcatttaatggagaacatatgggtttacttcgtggatatagtaaacgaaaattatctagttataaaaatcctgcatctagatcatcaaatgcaacggttgcacaattattaaatccaccacaaaaaggaaagaaagctgtagaatttgattttaataaatatatggatagtaaagctatgacagttaatgaagcgttaaataaggctattcctctaagatatccacaaaaaatttatgaaagtatgcgttattcattattagcaggtggtaaacgagtacgtcctgtcttatgcatagcagcttgtgaattagttggtggtactgaagaactagctattccaacagcgtgtgctatcgaaatgattcacactatgtcattgatgcatgatgatttaccatgtattgataatgatgatttaagaagaggtaaaccaacaaatcataaaatctttggtgaagacacagctgttacagcaggcaacgcgttacattcatatgcattcgagcatatagctgttagtactagtaagactgtcggagcggatagaatattacgtatggtatcagaattaggaagagctactggttcagaaggtgttatgggaggtcaaatggtagatattgcgtcagaaggtgatccatcaatcgatttacaaacactagagtggattcacattcataaaacagcgatgttgttagaatgttcagtagtttgtggagcgattataggtggtgcaagtgaaatcgtaattgaaagagcgcgtcgttacgcaagatgtgttggcttattatttcaagttgttgatgatattttagatgtaacaaaatctagtgatgaattaggtaaaacagcaggtaaagacttaatatctgataaagcaacatacccaaaattgatgggattggagaaagcaaaagaattctctgatgaattgcttaatagagcaaaaggtgaattatcatgttttgatccagttaaagcagctccacttcttggattagcagattatgtagcattcagacaaaatcggatgtaaacagagaaacgtcacactggctcaccttcgggtgggcctttctgcgtttatagcctaatacacctgttcgtggagaatataaaaagccagattattaatccggcttttttattattt

SEQ ID NO: 14 - protein, 361 aa, synthetic construct
MAGTAWDTDIPPLISVRDDVDAVLEDFVDMQERAALGPEMAPLFTTVRRLLFSNGKRLRPLLCVAGWQAAGAPGDSHPVLRVAAALELFQTFALIHDDVMDDSDTRRGRPSAHRALAAEYLSGGGRLSKAEAHGRGAAILLGDLLLVWSDEMLAGAGLGARAQARVLPLVDSMRAELVYGQYLDLLSTDRMSGNVEAALRVTRYKTGKYTVERPLHLGVCLAGGDPRLLQTCTDFAIPLGEAFQLRDDLLNVFGDPVQTGKPVGDDIRAGKATVLLALAVQDASGADLKLLSTLVGSPELGSEDIDRVRTVMETTGARQRVEKMITARRDTALATLEEADLPRDVAKTLRQIVWMATERQS

SEQ ID NO: 15 - protein, 533 aa, synthetic construct
MLEVPAQPTPAPREAEAAALLAATVADPWGLVAPSVYDTARLVSLAPWLDGHRERLGYLVKEQNQDGSWGAPDGYGLVPTLSAVEALLTELARTDSGAPHPPHDDLAAACAGGLGALQDGLLAGPVPDTIGVEFVAPSLLADINTRLAALTEQAPGKLGAWSGTTLTSPAPDLDGALLAGVREMTEQAPLPEKLWHTLEAITRDGTRGARPHEGAPPHNGSVGCSPAATAAWLGASPDPAAPGVAYLRDVQARFGGPVPSITPIVYFEQAWVLNSLAASGLRYEAPAALLDSLEAGLTDEGIAAAPGLPSDSDDTAAVLFALAQHGRTHRPDSLMHFRRDGYFSCFGVERTPSTSTNAHILEALGHHVTVRPDDAGRYGAEIRMISDWLLDNQLPDGSWMDKWHASPYYATACCALALAEFGGPSARAAVDRAAAWALETQRADGSWGRWQGTTEETAYMVQLLMRTRTPGSPGTVARSAARGCDALLAHDDPASYPGLWHDKDIYAPVTVIRAARLAALALGGAESAASGGA

SEQ ID NO: 16 - protein, 300 aa, synthetic construct
MIQTERAVQQVLEWGRSLTGFADEHAVEAVRGGQYILQRIHPSLRGTSARTGRDPQDETLIVTFYRELALLFWLDDCNDLGLISPEQLAAVEQALGQGVPCALPGFEGCAVLRASLATLAYDRRDYAQLLDDTRCYSAALRAGHAQAVAAERWSYAEYLHNGIDSIAYANVFCCLSLLWGLDMATLRARPAFRQVLRLISAIGRLQNDLHGCDKDRSAGEADNAVILLLQRYPAMPVVEFLNDELAGHTRMLHRVMAEERFPAPWGPLIEAMAAIRVQYYRTSTSRYRSDAVRGGQRAPA

SEQ ID NO: 17 - DNA, 11551 bp, synthetic construct
  source 1..11551: mol_type = other DNA
cggtggaaacgaggtcatcatttccttccgaaaaaacggttgcatttaaatcttacatatgtaatactttcaaagactacatttgtaagatttgaagctgtcaccggatgtgctttccggtctgatgagtccgtgaggacgaaacagcctctacaaataattttgtttaattaggaggatgattatttatgctagaggtaccagctcaacctacacctgctccaagagaggctgaggcagccgctttgcttgccgcaacagtagcagacccgtggggtttggtggccccgtctgtatatgatacggcacgattagtcagtttagctccttggttagacgggcatagagagagattaggatatttagtaaaggagcaaaaccaagacggtagttggggagcaccagatggatacggattagtaccgacactatcagcagtagaggctttattaacagagttggctagaacagactcaggcgcaccacacccacctcatgacgacctagcagccgcatgtgctggtggcttaggggctctacaagacggtttgttagcaggtcctgtgccagacacaattggagtagaattcgtggcaccatctcttttagcagacattaatactagactagcagccttaactgagcaggcaccgggtaagttaggtgcttggtcaggcactacattaacttctcctgcaccagacttagacggtgcgttactagcaggtgtacgagaaatgacagaacaagcacctttacctgaaaagctttggcacactttagaggcaattacacgtgacggcacaagaggcgctcgtccacacgagggagctccacctcataacggtagtgtgggatgctcacctgctgcaactgcggcttggctaggagcctcaccagacccagctgcacctggggttgcttatttaagagacgtccaagcgagattcggtggacctgtcccatctataacacctatagtttatttcgaacaagcttgggttttaaactctcttgcagctagtggtttacgttacgaggcaccagctgctcttctagattcacttgaggcgggacttacagacgagggtatagcagctgctcctggtcttccatcagattcagatgacacagcggctgtcttatttgcattagcacaacatggacgaactcaccgtccggatagtctaatgcattttagaagagacggttacttcagttgcttcggagtggaacgtactccgagtacatctactaatgcacacattttagaggctttaggccatcacgtgactgttagaccagatgacgcaggacgatacggagctgagattcgaatgatcagtgattggttactagacaaccagcttcctgacggttcatggatggacaagtggcatgctagtccttactatgctacagcttgttgcgcattagccttagcagagttcggtggtccatcagcacgagcagctgtagacagagcagctgcatgggcgttagagacgcagcgagcagacggttcatggggacgttggcaaggtacgactgaggagactgcttatatggtacaactacttatgcgtacacgaactccaggatctcctggaacagtagcacgttcagccgctagaggttgcgacgcgttacttgcgcacgacgaccctgcatcatatcctggtttatggcacgacaaggatatatacgcaccagtcactgttataagagcagcgcgattggcagctttagcgcttggtggagctgaaagtgcggcttctggtggagcttgattaggaggatgattatttatgatacaaacagaaagagcagttcaacaagtacttgagtggggtagatctttaacgggctttgctgacgagcatgcggttgaagccgtgcgtggtggacaatatatcttgcagagaatacacccaagtttgagaggaacttctgcgcgtactggacgtgaccctcaggacgagacattaatagtaacattttaccgtgagttggcattacttttctggttagatgattgcaatgacttaggccttatatcacctgagcagttggctgctgtggagcaagctcttggtcagggagtcccttgtgcactaccaggtttcgagggatgcgctgttcttcgtgcctcattagcaacattggcatacgaccgtcgtgattacgcacagttattagatgacactcgttgctattcagcagcattacgtgctggacatgcgcaggcagttgcagctgaaagatggagttacgcagagtatttacataacggtattgatagtatcgcttatgcgaatgttttctgttgcctttcattgttatggggattagacatggcaacattaagagcacgaccagccttccgtcaggtgttaagacttatttcagcgattggaagattacagaacgatttgcatggatgtgataaggatcgtagtgcaggagaagctgacaacgcggtaatattattactacaacgttatcctgctatgccagtggttgagttcttgaatgacgagcttgctggtcacacacgtatgttacatagagtaatggcagaagaaagattccctgctccttggggtccattgattgaggcaatggctgcaatacgtgtccagtactacagaactagtacatcaagataccgttcagacgctgttcgaggcggtcaacgtgcacctgcttaattaggaggatgattatttatggctgaagcttatattgttgaagcagttagaacgcctgtcggtagaagacgaggtggattagctggcgtacacccagctgatttaggagcacatgctttaacagctctagttgcacgttcaggaattgatcctgcagcggttgaagatgttgtatttggttgcttagatacagttggtccacaggctggtgatattgcaagaacttgttggttagcagcaggtcttcctgaagaagtaccaggagtgacagtagatagacaatgtggaagttcacaacaagctgtgcattttgctgcacaagctgttctatcaggtactcaggatttggtggtcgctggaggagttcaaaatatgtcacaaattccaatagcatttgcttctagacaagcagctgaacctttaggtttaacgcaaggaccattcgcaggttctgaaggttggcgtgctcgatatggtgatcttccagttaatcaatttcatggcgcagaactaattgcggcaaagtggggaattacacgacgtgatcaagaagaatttgcattaagatcacatagaagagcggtacgtgcatctgacgaaggtagatttgaccgagaaacggttgcgtatggtaaagtcactgcggatgagggaccacgtagagatacaagtttagaaaaaatggcggctttagcaccagtaattgaaggtggaacgattacagcagcatgttcaagtcaagtaagtgatggcgcagctgctatgttattggcttcagaacgtgctttgagagaacatggattaacgcctagagcacgtgtgcatcatctaagtgtaagaggagaagatccaattagaatgttatctgcacctatccctgcaacagcacacgcattaaaaaaaactgctttagcaattgatgatatagatttagtagaaatcaatgaagcgttcgcacctgttgtacttgcatggttaaaagagactggagcagatcctgaaaaagtgaatgtcaatggtggcgcaatcgctttaggacatcctttaggtgctacaggtgttagattgatgacgacattattacacgagttagaaagaactggtggtcgttttggtttacaaacaatgtgtgaaggaggaggccaggcgaatgttacgatcatagaaagactttagttaggaggatgattatttatgactataggtatagacaaaataggtttcgctacatctcaatatgttttaaaactagaagatttagcattggcacgtcaagtagatccagcaaagttttctcaaggtttattaattgaatcattttctgtagcacctatcacagaagatattattacgttagcagcttctgcagcggaccaaatcctaactgatgaagatcgtgcaaaaatagacatggtaatattagcaactgaaagtagtactgaccaatcaaaagcaagtgctatttatgttcaccatcttgtgggcattcaaccatttgcacgaagttttgaagtaaagcaagcttgctacagtgctacagcagcattagattatgcaaaattacatgtagcaagtaaaccagatagtagagtattagtgatagcttcagacattgctcgttatggagtaggatcaccaggagaaagtacacaaggaagtggtagtattgcattgttggtgacagcaaatccacgtatcttagcattaaataaagataatgttgctcaaacacgtgatataatggatttctggcgaccaaattactcatttacaccatacgttgatggtatttatagtactaaacaatatcttaattgcttagagactacatggcaagcttatcagaaacgagaaaatttacaattaagtgattttgctgcagtctgttttcatatcccttttcctaaattagcattaaagggtttaaataacattatggataatactgtccctccagagcatagagaaaaactaattgaagcgttccaagcatcaattacttattctaaacaaattggcaatatttatacaggaagtctttacttaggattattgagtcttttagaaaatagtaaagttttacagtctggtgacaagatcggttttttctcatatggctcaggagcagtctctgaattttattcaggccaacttgttgcaggttatgataaaatgttaatgacaaatcgacaagcacttttggatcaaagaactagattgtcagtttctaaatacgaagatttattttatgaacaagttcaacttgatgataatggtaatgctaattttgatatatatctaacaggtaaatttgctcttacagcaatcaaagagcaccaaagaatttaccacactaatgataaaaattaattaggaggatgattatttatgactaaaacgaatttaaattggtctggtttctctaaaaaaacatttgaagagcgtttacaattaatagaaaaatttaaattactaaatgcagaaaacttaaatcaattaaaaacagatgttttattacctattcaaacggcgaaccagatgacagagaatgtgttaggtcgtttagcattacctttttcaatagcgccagactttttagttaatggttctacataccaaatgccatttgtcacagaagagccttctgtggtagctgcagcaagttttgctgcaaagttaataaaaagaagtggtggatttaaagcgcaaacacttaatcgtcaaatgatcggccaaatcgtgttatacgatattgatcaaatcgataatgcgaaagcagctatcttacataaaacgaaaaaattgatagctttggctaacaaagcatatccatcaattgttaaacgaggtggtggcgcaagaactattcatttggaagagaaaggtgaattcttaatattttatttaacggtagacacacaagaagcaatgggtgctaatatggtcaatacaatgatggaagctttggttccagacttaactcgtttgagtaagggacattgtttaatggcaatattatcaaattatgctacagaatcactagttacaacatcttgcgaaataccagttagattattagatcgtgacaaaacaaaatctttacaattagcacaaaaaattgaattagcaagtcgtctagctcaagtggatccttatcgtgcaacaacacataataaaggtatttttaatggtatagacgctgtcgtcattgctactggtaacgactggcgtgcaattgaagcaggtgcacatgcatatgcttcacgaaatggtagttatcaaggcttaagtcaatggcattttgatcaagacaaacaggttttattaggtcaaatgacattacctatgccaattgcttcaaaaggaggttcaataggcttaaatcctactgttagtattgcacatgatttattaaatcaaccagatgctaaaacactagctcaacttattgcaagtgtaggtttagcacaaaactttgcagcactaaaagcgttaactagttcaggtatccaagcaggtcatatgaaattacacgcgaaatctcttgctttattggcaggcgctactcaagatgaaattgcaccattggtaaacgctttgcttgctgataaaccaattaatctagaaaaagctcacttttatttatcacaactacgtcaaagttgagtcgacctgcaggcatgcaagcttggctgttttggttaggaggatgattatttatgaacgagaatataggttatggaaaagcacattctaaaattattttaataggtgaacatgcagtagtatatggctatccagcaattgctcttcctcttacagatatagaagtagtgtgtcatatttttccagcggataaaccactagtttttgacttctatgacactttaagtacagcaatatatgcggcacttgattatcttcaaagattgcaagaaccaatagcttacgaaatagttagtcaagtaccacagaagcgtggcatgggtagtagtgctgctgttagtattgcagcgattagagctgttttttcttattgtcaagagcctttaagtgacgatttacttgaaatcctagtaaataaagcagaaataattgcacacacaaatccttcaggattagatgctaaaacatgcttatctgatcacgctataaaatttattagaaatataggttttgagacaatcgaaattgctttaaatggttatctaattattgctgatacgggtattcatggtcatacaagagaagctgttaataaagtcgctcagtttgaagaaacaaatttaccatatttagcaaaacttggtgcattaacacaagcattagaaagagctatcaaccaaaaaaataaggtagcaattggacaattaatgacacaagctcatagtgcattgaaagcaattggtgtatctatctctaaagctgatcaattagtcgaagcagcattacgagcaggtgcattaggtgctaaaatgacaggtggtggtctaggtggttgtatgattgctttagcagatacaaaagatatggcagaaaaaatttcacatagattaaaggaggaaggagcggtaaatacatggattcagatgttatagttaggaggatgattatttatgagtaactactgtgtacaaacgggcggtaaattatatttaacaggagaatatgcaatacttattccaggtcaaaaagctttaattcactttattccattaatgatgactgctgaaatcagtccagctgcacatattcaacttgcttcagatatgttttcacacaaagcgggtatgacacctgatgcaagttatgctttaattcaagcaacagttaaaacatttgcagattacctaggacaaagtattgatcaattagaacctttcagtcttattatcacgggtaaaatggaacgtgatggaaaaaaattcggtataggaagttcaggatctgttactttattaactttaaaagcattgtcagcttattatcaaatcacattaacaccagaattgttatttaaattagctgcatatactttgctaaaacagggagataacggttctatgggagacatagcgtgcattgcttaccaaacattagttgcatatacatcttttgatcgtgaacaagtgtcaaattggttacaaactatgccattaaaaaaacttttagtcaaagattggggatatcatattcaagtaattcagcctgctttgccttgtgatttcttagttggttggactaagatacctgcaatttcacgtcaaatgattcagcaagtaacggcttctattacacctgcgtttttacgtacgtcatatcaattaacacaatctgctatggtggctttacaagaaggccataaagaagaattaaaaaaatcattggctggagcttcacacttattaaaagaattacaccctgctatttatcatcctaaattggttacattagttgctgcttgtcaaaaacaggatgcagtagcaaaaagtagtggaagtggtggaggcgactgcggtattgctcttgcatttaatcaagatgcgcgtgatacattaatttcaaaatggcaagaagctgatatagctcttttataccaagaaagatggggagagaatgactagttaggaggatgattatttatggatcctaatgtaatcacggttacttcatatgcaaatatagcaataattaaatactggggtaaagaaaatcaagctaaaatgatcccttctacatcttctatttcattgacattagaaaatatgttcactactacttcagtgtcatttttaccagacactgcaacttcagatcaattttatataaacggtgtgttacaaaatgatgaagaacatacgaaaatatcaacaattattgatcaattccgtcaaccaggacaagcttttgtaaaaatggaaactcaaaataacatgcctacagctgcaggattatcatcatcatcttcaggtttgtctgcattagtaaaggcttgtgatcaattgtttgatacacagctagatcaaaaagcattagcacaaaaagcaaaatttgcgtctggaagttcatcacgttctttctttggtcctgtcgcggcatgggataaagattctggcgcaatatataaagttgaaacggacttgaaaatggcgatgattatgttagttttaaacgcagcaaaaaaacctatttcatcacgtgaaggtatgaaattatgtcgtgatacatcaacaacatttgatgaatgggttgaacagtcagcaattgactaccaacatatgttgacatatttaaaaacaaacaacttcgaaaaagttggccaattaacagaagcgaatgcattggctatgcatgcaactacaaagacagcgaatcctccttttagttacctaactaaagaatcttatcaagctatggaagctgtaaaagaattgcgtcaagaaggttttgcatgctattttacaatggatgcaggacctaatgtcaaagtactttgtttagagaaagatttagcacaattggcagagagattgggaaaaaattatcgaattatagtatcaaaaacaaaagacttgccagacgtttaattaggaggatgattatttatgactaatagaaaagatgatcatattaagtatgctcttaaataccaatcaccttataacgcttttgatgatattgaattaattcatcatagtctaccttcttatgatcttagtgacattgatttatcaactcactttgcaggacaagattttgactttccattttatattaacgctatgacaggaggatctcaaaaaggtaaagctgttaatgaaaaattagcaaaagttgcagctgcaacaggtattgtaatggtaacaggatcttatagtgcagctttgaaaaatccaaatgatgattcttatagattacacgaggttgctgacaatcttaaattagctactaacattggtttagacaagcctgttgctttaggtcagcagacagtgcaagaaatgcaacctttgtttcttcaagtacatgttaacgttatgcaagaattgttaatgccagaaggtgaaagagtttttcatacttggaaaaaacatctagctgaatatgctagtcaaataccagtcccagtaattcttaaagaagtaggctttggtatggatgttaactctattaaattagcacatgatttgggtattcaaactttcgatataagtggtagaggtggaactagtttcgcttatatagaaaatcaacgtggtggcgatagatcatacttgaatgactggggtcaaactacagttcagtgtcttttaaatgcacaaggcctaatggatcaagtcgaaatattagcatcaggtggtgtacgtcatcctcttgatatgattaaatgttttgttttaggcgctcgtgcagtaggattatctagaacggtgttggaattagttgaaaaatatccaactgagagagtaatcgctatagtaaatggttggaaggaagaattgaaaataattatgtgtgcattggattgcaaaacaataaaagagctaaaaggcgtggattatttattatacggacgacttcaacaagtaaattaagcgaagacttaatcattaggaggatgattatttatggcaggaacggcatgggatactgatataccaacgcttatttcagtcagagatgatgtggatgcagtacttgaggactttgttgatgttcaagagagagcggcacacggaccagagatggctccattgttcacaacggctagaagacttttgtttagtaacggtaaaagactaagaccattattatgcgtggcggggtggcaagcagctggagctccaggtgacccgcatgctgtcttgcgtgttgcagcagcattagaactattccagacattcgcgctaatacatgacgacgtaatggatgacagtgatactagaagaggtagacctagtgcacatagagctttggcagcagagtacatgagtggcggtggacgattaagtaaggctgaagcgcatggcagaggagctgccattttgttaggtgaccttttattagtctggtctgacgaaatgcttgctggtgcgggccttgaggcgagagcacgagctagagtacttccattagttgactctatgagagctgagttagtatacggtcagtacttagatttattatctactgatagaatgtcaggtgacgttgaagcggcgctaagagtagcaagatataagacaggcaagtatacagttgaaagaccacttcatttgggtgttagtttagcgggcggtgacccacgattattacagacgtgtacggattttgcattgccattaggtgaagcattccaacttcgtgatgatcttttaaacgtctttggggatccagttcgtactggtaagccggttggtgacgatatacgtgaaggtaaagctacagtattgttagctcttgctatacaggacgcgtcagaagctgacttaaaattattaggtactttagttggtagtcctgagttaggtagtgaagatatagaccgattcagaacagtaatggagactacaggtgcacgtcaacgtgtagaggacatgataatcgcacgtagagacactgcacttgcaactttggaagaagcggatttaccacaggatgtagcagagacattaagacagatcgtttggatggcgacggagagacaatcataacggatgtaaacagagaaacgtcacactggctcaccttcgggtgggcctttctgcgtttatagcctaatacacctgttcgtggagaatataaaaagccagattattaatccggcttttttattattt

SEQ ID NO: 18 - protein, 512 aa, synthetic construct
MPAMSDADRIAALLKDRAADPVTKFSPSPYETGQFLRISERADVGTPQIDYLLATQRPDGLWGSVGFELVPTLGAVAGLSSRPEYADRAGVTDAVARACEKLWELALGEGGLPKLPDTVASEIIVPSLIDLLSEVLQRHRPAVGGKAGQEQEFPSPPGAKPELWRQLSDRIARGQAIPKTAWHTLEAFHPLPKQFAATVTPAADGAVTCSPSSTAAWLSAVGTDAGASTRAYLDEAQSRYGGAIPMGSSMPYFEVLWVLNLVLKYFPDVPIPREIIEEIAAGFSDSGIGGGPGLPPDGDDTAYANLAGDKLGAPTHPEILMKFWAEDHFVSYPGEQTPSETVNAHALEYLNHLRMRRGITEFGAVEDACAEWVISQQTEDGCWYDKWNVSPYYSTAACVEALLDARKQDEPQLDSLRRAREWLLRHQTDSGGWGMAEPSPEETAYAVLALDLFASRGGEGAEECAAAISRAKEFFTDESRENPPLWMGKDLYTPFRIVDVTVMCGRAVVGRY

SEQ ID NO: 19 - protein, 311 aa, synthetic construct
MPDAIEFEHEGRRNPNSAEAESAYSSIIAALDLQESDYAVISGHSRIVGAAALVYPDADAETLLAASLWTACLIVNDDRWDYVQEDGGRLAPGEWFDGVTEVVDTWRTAGPRLPDPFFELVRTTMSRLDAALGAEAADEIGHEIKRAITAMKWEGVWNEYTKKTSLATYLSFRRGYCTMDVQVVLDKWINGGRSFAALRDDPVRRAIDDVVVRFGCLSNDYYSWGREKKAVDKSNAVRILMDHAGYDESTALAHVRDDCVQAITDLDCIEESIKRSGHLGSHAQELLDYLACHRPLIYAAATWPTETNRYR

SEQ ID NO: 20 - DNA, 11521 bp, synthetic construct
  source 1..11521: mol_type = other DNA
cggtggaaacgaggtcatcatttccttccgaaaaaacggttgcatttaaatcttacatatgtaatactttcaaagactacatttgtaagatttgaagctgtcaccggatgtgctttccggtctgatgagtccgtgaggacgaaacagcctctacaaataattttgtttaattaggaggatgattatttatgccagctatgagtgatgcggacagaattgcagcattgttaaaagatcgagctgcagatccagtcacgaagttttcaccttctccatacgaaacgggtcaatttttgcgaatttcagaacgtgcagatgtgggcacgcctcaaattgattatcttttagcaacacaacgtccagacggattatggggtagtgttggcttcgaattagttccaactcttggtgctgttgctggtctttcttcaagacctgagtatgctgatcgtgctggtgtaacagacgcagttgctcgagcttgtgaaaagttatgggaattagcgttgggcgaaggaggattaccaaaattgcctgatacagttgcgtcagaaatcattgtaccttctcttattgacttgttaagtgaagttttacaaagacatcgtccagcagtgggaggtaaagcaggacaagaacaagagtttccatcacctcctggagcaaaaccagagttatggagacaattaagtgatcgtatcgcacgtggacaagcaatcccgaaaactgcttggcacacattagaagcttttcatccattacctaagcagtttgcagctactgtaacaccagcagctgatggtgctgtaacatgcagtccttcatctactgcagcgtggctatcagctgttggtacagatgctggtgcgtctacacgagcttatttggatgaagcacaatctagatatggtggtgcgattcctatgggatcaagtatgccatattttgaagtactatgggtacttaatctagtcttaaaatattttcctgatgttcctatacctagagaaataattgaagaaattgctgcgggtttcagtgattctggtattggtggtggtccgggcttaccaccagatggtgatgacacggcatatgcgaacctagcaggagataaattaggagcacctacacatccggaaatattaatgaaattctgggcggaagatcactttgttagttatccaggtgaacaaacaccatctgaaactgtaaatgcacatgcgctagaatatttaaatcatcttcgtatgcgaagaggtataacagaattcggtgcagtggaagatgcgtgtgctgaatgggtgatatcacaacagactgaggacggctgttggtacgataaatggaatgtctctccttactattcaactgcagcatgtgtagaggctttattagatgcacgtaaacaagatgaaccgcaattagacagtttacgtcgagctagagagtggcttttaagacatcaaactgatagtggaggatggggtatggctgagccgagtcctgaagaaacagcttatgctgtactagcgcttgatttatttgcaagtagaggtggcgaaggagctgaagaatgtgcagcagctatttcacgtgcaaaagaattttttactgatgaaagtagagaaaatcctccattatggatgggcaaagatttatatacaccatttcgtattgttgatgtaactgttatgtgtggaagagcagtcgttggtcgttattaattaggaggatgattatttatgccagacgctatagaatttgagcatgaaggtagaagaaacccaaactcagctgaagcagaatcagcttatagttctattattgctgcgttggatttacaagaatcagattatgctgttatttcaggtcatagtcgtattgtgggcgctgcagctttagtttatcctgatgctgatgcggaaactttacttgctgcaagtttatggacggcttgtttaattgtgaatgatgacagatgggattatgtccaagaggatggaggccgtttagcacctggtgaatggttcgatggtgtaacggaagtagttgatacttggcgaacagctggtccaagactacctgatccattctttgaattggttcgaacaacaatgtctagattggacgcggctcttggagcagaggcagcagatgaaataggtcacgaaattaaacgagcgataacagcgatgaaatgggaaggagtttggaatgaatatacgaagaaaactagtttagcgacttatttaagttttcgtcgtggatattgtacaatggatgttcaagtggtattagataaatggattaatggtggtagatcttttgctgctttacgagatgacccggtcagaagagcaatcgatgacgttgtagtacgttttggatgcttatctaatgattactactcatggggccgtgaaaagaaagcagtagataaaagtaatgcagttagaatccttatggatcatgcaggttatgatgaatcaactgcgttggctcatgttcgtgacgattgtgtacaagcaattactgatcttgattgtattgaagaaagtataaaacgttctggacacctaggtagtcatgcacaggagttactagattatttagcatgtcatcgtccattaatctacgcagcagcaacatggcctacagaaacaaatcgttatagataattaggaggatgattatttatggctgaagcttatattgttgaagcagttagaacgcctgtcggtagaagacgaggtggattagctggcgtacacccagctgatttaggagcacatgctttaacagctctagttgcacgttcaggaattgatcctgcagcggttgaagatgttgtatttggttgcttagatacagttggtccacaggctggtgatattgcaagaacttgttggttagcagcaggtcttcctgaagaagtaccaggagtgacagtagatagacaatgtggaagttcacaacaagctgtgcattttgctgcacaagctgttctatcaggtactcaggatttggtggtcgctggaggagttcaaaatatgtcacaaattccaatagcatttgcttctagacaagcagctgaacctttaggtttaacgcaaggaccattcgcaggttctgaaggttggcgtgctcgatatggtgatcttccagttaatcaatttcatggcgcagaactaattgcggcaaagtggggaattacacgacgtgatcaagaagaatttgcattaagatcacatagaagagcggtacgtgcatctgacgaaggtagatttgaccgagaaacggttgcgtatggtaaagtcactgcggatgagggaccacgtagagatacaagtttagaaaaaatggcggctttagcaccagtaattgaaggtggaacgattacagcagcatgttcaagtcaagtaagtgatggcgcagctgctatgttattggcttcagaacgtgctttgagagaacatggattaacgcctagagcacgtgtgcatcatctaagtgtaagaggagaagatccaattagaatgttatctgcacctatccctgcaacagcacacgcattaaaaaaaactgctttagcaattgatgatatagatttagtagaaatcaatgaagcgttcgcacctgttgtacttgcatggttaaaagagactggagcagatcctgaaaaagtgaatgtcaatggtggcgcaatcgctttaggacatcctttaggtgctacaggtgttagattgatgacgacattattacacgagttagaaagaactggtggtcgttttggtttacaaacaatgtgtgaaggaggaggccaggcgaatgttacgatcatagaaagactttagttaggaggatgattatttatgactataggtatagacaaaataggtttcgctacatctcaatatgttttaaaactagaagatttagcattggcacgtcaagtagatccagcaaagttttctcaaggtttattaattgaatcattttctgtagcacctatcacagaagatattattacgttagcagcttctgcagcggaccaaatcctaactgatgaagatcgtgcaaaaatagacatggtaatattagcaactgaaagtagtactgaccaatcaaaagcaagtgctatttatgttcaccatcttgtgggcattcaaccatttgcacgaagttttgaagtaaagcaagcttgctacagtgctacagcagcattagattatgcaaaattacatgtagcaagtaaaccagatagtagagtattagtgatagcttcagacattgctcgttatggagtaggatcaccaggagaaagtacacaaggaagtggtagtattgcattgttggtgacagcaaatccacgtatcttagcattaaataaagataatgttgctcaaacacgtgatataatggatttctggcgaccaaattactcatttacaccatacgttgatggtatttatagtactaaacaatatcttaattgcttagagactacatggcaagcttatcagaaacgagaaaatttacaattaagtgattttgctgcagtctgttttcatatcccttttcctaaattagcattaaagggtttaaataacattatggataatactgtccctccagagcatagagaaaaactaattgaagcgttccaagcatcaattacttattctaaacaaattggcaatatttatacaggaagtctttacttaggattattgagtcttttagaaaatagtaaagttttacagtctggtgacaagatcggttttttctcatatggctcaggagcagtctctgaattttattcaggccaacttgttgcaggttatgataaaatgttaatgacaaatcgacaagcacttttggatcaaagaactagattgtcagtttctaaatacgaagatttattttatgaacaagttcaacttgatgataatggtaatgctaattttgatatatatctaacaggtaaatttgctcttacagcaatcaaagagcaccaaagaatttaccacactaatgataaaaattaattaggaggatgattatttatgactaaaacgaatttaaattggtctggtttctctaaaaaaacatttgaagagcgtttacaattaatagaaaaatttaaattactaaatgcagaaaacttaaatcaattaaaaacagatgttttattacctattcaaacggcgaaccagatgacagagaatgtgttaggtcgtttagcattacctttttcaatagcgccagactttttagttaatggttctacataccaaatgccatttgtcacagaagagccttctgtggtagctgcagcaagttttgctgcaaagttaataaaaagaagtggtggatttaaagcgcaaacacttaatcgtcaaatgatcggccaaatcgtgttatacgatattgatcaaatcgataatgcgaaagcagctatcttacataaaacgaaaaaattgatagctttggctaacaaagcatatccatcaattgttaaacgaggtggtggcgcaagaactattcatttggaagagaaaggtgaattcttaatattttatttaacggtagacacacaagaagcaatgggtgctaatatggtcaatacaatgatggaagctttggttccagacttaactcgtttgagtaagggacattgtttaatggcaatattatcaaattatgctacagaatcactagttacaacatcttgcgaaataccagttagattattagatcgtgacaaaacaaaatctttacaattagcacaaaaaattgaattagcaagtcgtctagctcaagtggatccttatcgtgcaacaacacataataaaggtatttttaatggtatagacgctgtcgtcattgctactggtaacgactggcgtgcaattgaagcaggtgcacatgcatatgcttcacgaaatggtagttatcaaggcttaagtcaatggcattttgatcaagacaaacaggttttattaggtcaaatgacattacctatgccaattgcttcaaaaggaggttcaataggcttaaatcctactgttagtattgcacatgatttattaaatcaaccagatgctaaaacactagctcaacttattgcaagtgtaggtttagcacaaaactttgcagcactaaaagcgttaactagttcaggtatccaagcaggtcatatgaaattacacgcgaaatctcttgctttattggcaggcgctactcaagatgaaattgcaccattggtaaacgctttgcttgctgataaaccaattaatctagaaaaagctcacttttatttatcacaactacgtcaaagttgagtcgacctgcaggcatgcaagcttggctgttttggttaggaggatgattatttatgaacgagaatataggttatggaaaagcacattctaaaattattttaataggtgaacatgcagtagtatatggctatccagcaattgctcttcctcttacagatatagaagtagtgtgtcatatttttccagcggataaaccactagtttttgacttctatgacactttaagtacagcaatatatgcggcacttgattatcttcaaagattgcaagaaccaatagcttacgaaatagttagtcaagtaccacagaagcgtggcatgggtagtagtgctgctgttagtattgcagcgattagagctgttttttcttattgtcaagagcctttaagtgacgatttacttgaaatcctagtaaataaagcagaaataattgcacacacaaatccttcaggattagatgctaaaacatgcttatctgatcacgctataaaatttattagaaatataggttttgagacaatcgaaattgctttaaatggttatctaattattgctgatacgggtattcatggtcatacaagagaagctgttaataaagtcgctcagtttgaagaaacaaatttaccatatttagcaaaacttggtgcattaacacaagcattagaaagagctatcaaccaaaaaaataaggtagcaattggacaattaatgacacaagctcatagtgcattgaaagcaattggtgtatctatctctaaagctgatcaattagtcgaagcagcattacgagcaggtgcattaggtgctaaaatgacaggtggtggtctaggtggttgtatgattgctttagcagatacaaaagatatggcagaaaaaatttcacatagattaaaggaggaaggagcggtaaatacatggattcagatgttatagttaggaggatgattatttatgagtaactactgtgtacaaacgggcggtaaattatatttaacaggagaatatgcaatacttattccaggtcaaaaagctttaattcactttattccattaatgatgactgctgaaatcagtccagctgcacatattcaacttgcttcagatatgttttcacacaaagcgggtatgacacctgatgcaagttatgctttaattcaagcaacagttaaaacatttgcagattacctaggacaaagtattgatcaattagaacctttcagtcttattatcacgggtaaaatggaacgtgatggaaaaaaattcggtataggaagttcaggatctgttactttattaactttaaaagcattgtcagcttattatcaaatcacattaacaccagaattgttatttaaattagctgcatatactttgctaaaacagggagataacggttctatgggagacatagcgtgcattgcttaccaaacattagttgcatatacatcttttgatcgtgaacaagtgtcaaattggttacaaactatgccattaaaaaaacttttagtcaaagattggggatatcatattcaagtaattcagcctgctttgccttgtgatttcttagttggttggactaagatacctgcaatttcacgtcaaatgattcagcaagtaacggcttctattacacctgcgtttttacgtacgtcatatcaattaacacaatctgctatggtggctttacaagaaggccataaagaagaattaaaaaaatcattggctggagcttcacacttattaaaagaattacaccctgctatttatcatcctaaattggttacattagttgctgcttgtcaaaaacaggatgcagtagcaaaaagtagtggaagtggtggaggcgactgcggtattgctcttgcatttaatcaagatgcgcgtgatacattaatttcaaaatggcaagaagctgatatagctcttttataccaagaaagatggggagagaatgactagttaggaggatgattatttatggatcctaatgtaatcacggttacttcatatgcaaatatagcaataattaaatactggggtaaagaaaatcaagctaaaatgatcccttctacatcttctatttcattgacattagaaaatatgttcactactacttcagtgtcatttttaccagacactgcaacttcagatcaattttatataaacggtgtgttacaaaatgatgaagaacatacgaaaatatcaacaattattgatcaattccgtcaaccaggacaagcttttgtaaaaatggaaactcaaaataacatgcctacagctgcaggattatcatcatcatcttcaggtttgtctgcattagtaaaggcttgtgatcaattgtttgatacacagctagatcaaaaagcattagcacaaaaagcaaaatttgcgtctggaagttcatcacgttctttctttggtcctgtcgcggcatgggataaagattctggcgcaatatataaagttgaaacggacttgaaaatggcgatgattatgttagttttaaacgcagcaaaaaaacctatttcatcacgtgaaggtatgaaattatgtcgtgatacatcaacaacatttgatgaatgggttgaacagtcagcaattgactaccaacatatgttgacatatttaaaaacaaacaacttcgaaaaagttggccaattaacagaagcgaatgcattggctatgcatgcaactacaaagacagcgaatcctccttttagttacctaactaaagaatcttatcaagctatggaagctgtaaaagaattgcgtcaagaaggttttgcatgctattttacaatggatgcaggacctaatgtcaaagtactttgtttagagaaagatttagcacaattggcagagagattgggaaaaaattatcgaattatagtatcaaaaacaaaagacttgccagacgtttaattaggaggatgattatttatgactaatagaaaagatgatcatattaagtatgctcttaaataccaatcaccttataacgcttttgatgatattgaattaattcatcatagtctaccttcttatgatcttagtgacattgatttatcaactcactttgcaggacaagattttgactttccattttatattaacgctatgacaggaggatctcaaaaaggtaaagctgttaatgaaaaattagcaaaagttgcagctgcaacaggtattgtaatggtaacaggatcttatagtgcagctttgaaaaatccaaatgatgattcttatagattacacgaggttgctgacaatcttaaattagctactaacattggtttagacaagcctgttgctttaggtcagcagacagtgcaagaaatgcaacctttgtttcttcaagtacatgttaacgttatgcaagaattgttaatgccagaaggtgaaagagtttttcatacttggaaaaaacatctagctgaatatgctagtcaaataccagtcccagtaattcttaaagaagtaggctttggtatggatgttaactctattaaattagcacatgatttgggtattcaaactttcgatataagtggtagaggtggaactagtttcgcttatatagaaaatcaacgtggtggcgatagatcatacttgaatgactggggtcaaactacagttcagtgtcttttaaatgcacaaggcctaatggatcaagtcgaaatattagcatcaggtggtgtacgtcatcctcttgatatgattaaatgttttgttttaggcgctcgtgcagtaggattatctagaacggtgttggaattagttgaaaaatatccaactgagagagtaatcgctatagtaaatggttggaaggaagaattgaaaataattatgtgtgcattggattgcaaaacaataaaagagctaaaaggcgtggattatttattatacggacgacttcaacaagtaaattaagcgaagacttaatcattaggaggatgattatttatggcaggaacggcatgggatactgatataccaacgcttatttcagtcagagatgatgtggatgcagtacttgaggactttgttgatgttcaagagagagcggcacacggaccagagatggctccattgttcacaacggctagaagacttttgtttagtaacggtaaaagactaagaccattattatgcgtggcggggtggcaagcagctggagctccaggtgacccgcatgctgtcttgcgtgttgcagcagcattagaactattccagacattcgcgctaatacatgacgacgtaatggatgacagtgatactagaagaggtagacctagtgcacatagagctttggcagcagagtacatgagtggcggtggacgattaagtaaggctgaagcgcatggcagaggagctgccattttgttaggtgaccttttattagtctggtctgacgaaatgcttgctggtgcgggccttgaggcgagagcacgagctagagtacttccattagttgactctatgagagctgagttagtatacggtcagtacttagatttattatctactgatagaatgtcaggtgacgttgaagcggcgctaagagtagcaagatataagacaggcaagtatacagttgaaagaccacttcatttgggtgttagtttagcgggcggtgacccacgattattacagacgtgtacggattttgcattgccattaggtgaagcattccaacttcgtgatgatcttttaaacgtctttggggatccagttcgtactggtaagccggttggtgacgatatacgtgaaggtaaagctacagtattgttagctcttgctatacaggacgcgtcagaagctgacttaaaattattaggtactttagttggtagtcctgagttaggtagtgaagatatagaccgattcagaacagtaatggagactacaggtgcacgtcaacgtgtagaggacatgataatcgcacgtagagacactgcacttgcaactttggaagaagcggatttaccacaggatgtagcagagacattaagacagatcgtttggatggcgacggagagacaatcataacggatgtaaacagagaaacgtcacactggctcaccttcgggtgggcctttctgcgtttatagcctaatacacctgttcgtggagaatataaaaagccagattattaatccggcttttttattattt

SEQ ID NO: 21 - DNA, 97 bp, synthetic construct
  source 1..97: mol_type = other DNA
aattttgtcaaaataattttattgacaacgtcttattaacgttgatataatttaaattttatttgacaaaaatgggctcgtgttgtacaataaatgt

SEQ ID NO: 22 - DNA, 239 bp, synthetic construct
  source 1..239: mol_type = other DNA
atttttaaagtatgtatacaaatgatgaataaattttggcgatataatgaaggatacagctcccataattggtaaagatactagatagattcatcgtaaaatcatgattttgccaaatttgcccttgaatattagtagcgttttctttacaatcgtaaatagtgtaaaaaagcgtgcaaacgcatgaatatcatctaaaggagagattcacatgggaaaactgtatgtatttgatcctc

SEQ ID NO: 23 - DNA, 89 bp, synthetic construct
  source 1..89: mol_type = other DNA
ttttcctcctttaaacaaaattatttgtagaggactgtttcggcccttttgggccatcgtcaggtcggatacacatccggcgacagtct

SEQ ID NO: 24 - DNA, 101 bp, synthetic construct
  source 1..101: mol_type = other DNA
ctcgagggtaaatgtgagcactcacaattcattttgcaaaagttgttgactttatctacaaggtgtggcataatgtgtgtaattgtgagcggataacaatt

SEQ ID NO: 25 - DNA, 56 bp, synthetic construct
  source 1..56: mol_type = other DNA
attttacatttttagaaatgggcgtgaaaaaaagcgcgcgattatgtaaaatataa